(19) United States
(12) Patent Application Publication
(10) Pub. No.: US 2015/0030693 A1
(43) Pub. Date: Jan. 29, 2015

(54) ANTI-TUMOR AQUEOUS SOLUTION, ANTI-CANCER AGENT, AND METHODS FOR PRODUCING SAID AQUEOUS SOLUTION AND SAID ANTI-CANCER AGENT

(71) Applicants: NATIONAL UNIVERSITY CORPORATION NAGOYA UNIVERSITY, Nagoya-shi, Aichi (JP); NU ECO ENGINERRING CO., LTD., Miyoshi-shi (JP)

(72) Inventors: Masaru Hori, Nagoya-shi (JP); Masaaki Mizuno, Nagoya-shi (JP); Fumitaka Kikkawa, Nagoya-shi (JP); Hiroaki Kajiyama, Nagoya-shi (JP); Fumi Utsumi, Nagoya-shi (JP); Kae Nakamura, Nagoya-shi (JP); Kenji Ishikawa, Nagoya-shi (JP); Keigo Takeda, Nagoya-shi (JP); Hiromasa Tanaka, Nagoya-shi (JP); Hiroyuki Kano, Miyoshi-shi (JP)

(73) Assignees: c/o NATONAL UNIVERSITY CORPORATION NAGOYA UNIVERSITY, Nagoya-shi (JP); NU ECO ENGINEEERING CO., LTD., Miyoshi-shi (JP)

(21) Appl. No.: 14/381,190

(22) PCT Filed: Feb. 26, 2013
(86) PCT No.: PCT/JP2013/001139

§ 371 Date: Aug. 26, 2014

(30) Foreign Application Priority Data
Feb. 27, 2012 (JP) 2012-039645

(51) Int. Cl. A61K 33/42; A61K 41/00; A61K 31/4172; B01J 19/08; A61K 33/00; A61K 31/198

(52) U.S. Cl. CPC A61K 33/42; A61K 33/00; A61K 31/198; A61K 31/4172; B01J 19/088; A61K 41/0023; B01J 2219/0809
USPC 424/605; 424/715; 514/563; 514/400; 514/567; 435/404; 204/165; 204/164

(57) ABSTRACT

An object of the present invention is to provide an antitumor aqueous solution and an anticancer agent, both of which can kill cancer cells while having virtually no effects on normal cells, and to provide methods for producing the antitumor aqueous solution and the anticancer agent. The method of the invention for producing an antitumor aqueous solution for killing cancer cells includes an aqueous solution preparation step of preparing an aqueous solution through addition, to water, of a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O); and a plasma irradiation step of irradiating the aqueous solution with atmospheric pressure plasma generated in a plasma generation region by means of a plasma generator.

### TECHNICAL FIELD

The present invention relates to an antitumor aqueous solution and an anticancer agent, and to production methods therefor. More particularly, the present invention relates to an antitumor aqueous solution and an anticancer agent, both of which can kill cancer cells, and to production methods therefor.

### BACKGROUND ART

Plasma technology has been applied to the fields of electricity, chemistry, and materials. In recent years, extensive studies have been conducted to apply plasma technology to the medical field. Charged particles (e.g., electrons or ions) are generated in plasma, and UV rays or radicals are also generated therein. It has been found that such radicals exhibit various effects on biological tissues (e.g., biological tissue sterilization).

For example, Patent Document 1 describes that plasma irradiation exhibits effects on blood coagulation (see Example 4 of Patent Document 1, paragraphs [0063]-[0068]), tissue sterilization (see Example 5 of Patent Document 1, paragraphs [0069]-[0074]), and leishmaniasis (see Example 6 of Patent Document 1, paragraphs [0075]-[0077]). Patent Document 1 also describes that plasma irradiation exhibits the effect of killing melanoma cells (malignant melanoma cells) (see Example 7 of Patent Document 1, paragraph [0078]).

### PRIOR ART DOCUMENT

### Patent Document

Patent Document 1: Japanese Kohyo Patent Publication No. 2008-539007

### SUMMARY OF THE INVENTION

### Problems to be Solved by the Invention

Generally, such cancer treatment is desirably carried out 1) to kill cancer cells, and 2) not to affect normal cells, for the following reason. Even if cancer cells can be killed in a patient, when many normal cells are also killed accordingly, a heavy physical burden is imposed on the patient. Therefore, demand has arisen for a therapeutic technique for selectively killing cancer cells. However, difficulty is encountered in selectively killing cancer cells. Patent Document 1 does not disclose the degree of the effect of plasma irradiation on normal cells.

The present invention has been accomplished for solving problems involved in the aforementioned conventional techniques. Accordingly, an object of the present invention is to provide an antitumor aqueous solution and an anticancer agent, both of which can kill cancer cells while having virtually no effects on normal cells. Another object of the present invention is to provide methods for producing the antitumor aqueous solution and the anticancer agent.

### Means for Solving the Problems

In a first aspect of the present invention, there is provided a method for producing an antitumor aqueous solution exhibiting an antitumor effect of killing cancer cells. The antitumor aqueous solution production method comprises an aqueous solution preparation step of preparing an aqueous solution through addition, to water, of a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O); and a plasma irradiation step of irradiating the aqueous solution with atmospheric pressure plasma generated in a plasma generation region by means of a plasma generator.

The antitumor aqueous solution produced through this production method kills cancer cells, but kills virtually no normal cells. Therefore, human cancer can be treated by bringing the antitumor aqueous solution into direct contact with cancer cells; by orally administering the antitumor aqueous solution to a patient; or by impregnating the periphery of a cancerous organ of a patient with the antitumor aqueous solution after, for example, laparotomy.

A second aspect of the present invention is drawn to a specific embodiment of the antitumor aqueous solution production method, wherein the plasma irradiation step employs a plasma density-time product of 1.2×10¹⁸ sec·cm⁻³ or more, the plasma density-time product being defined by the product of the plasma density in the plasma generation region and the time during which the aqueous solution is irradiated with the atmospheric pressure plasma.

A third aspect of the present invention is drawn to a specific embodiment of the antitumor aqueous solution production method, which further comprises a culture component addition step of adding a culture component to the aqueous solution irradiated with the atmospheric pressure plasma, the culture component addition step being carried out after the plasma irradiation step.

A fourth aspect of the present invention is drawn to a specific embodiment of the antitumor aqueous solution production method, wherein, in the aqueous solution preparation step, a culture solution is prepared, as the aqueous solution, through addition of a culture component to water. In the plasma irradiation step, the culture solution is irradiated with the atmospheric pressure plasma.

A fifth aspect of the present invention is drawn to a specific embodiment of the antitumor aqueous solution production method, wherein, in the plasma irradiation step, the aqueous solution is irradiated with the atmospheric pressure plasma while the level of the aqueous solution is adjusted so that the aqueous solution is not exposed to the plasma generation region.

A sixth aspect of the present invention is drawn to a specific embodiment of the antitumor aqueous solution production method, wherein the plasma generator includes a first electrode and a second electrode, the electrodes being located so as to face each other. In the plasma irradiation step, the aqueous solution is irradiated with the atmospheric pressure plasma while the first electrode and the second electrode are located outside the aqueous solution so that the aqueous solution is not provided between the electrodes.

A seventh aspect of the present invention is drawn to a specific embodiment of the antitumor aqueous solution production method, wherein the first electrode and the second electrode have facing surfaces. Each of the facing surfaces has small hollows.

An eighth aspect of the present invention is drawn to a specific embodiment of the antitumor aqueous solution production method, wherein the antitumor aqueous solution selectively kills cancer cells.

In a ninth aspect of the present invention, there is provided an antitumor aqueous solution exhibiting an antitumor effect of killing cancer cells. The antitumor aqueous solution is produced by dissolving, in water, a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O), to thereby prepare an aqueous solution, and irradiating the aqueous solution with atmospheric pressure plasma.

A tenth aspect of the present invention is drawn to a specific embodiment of the antitumor aqueous solution, wherein the atmospheric pressure plasma irradiation is carried out at a plasma density-time product of 1.2×10¹⁸ sec·cm⁻³ or more, the plasma density-time product being defined by the product of the plasma density in a plasma generation region of the atmospheric pressure plasma and the time during which the aqueous solution is irradiated with the atmospheric pressure plasma.

An eleventh aspect of the present invention is drawn to a specific embodiment of the antitumor aqueous solution, which is prepared by adding a culture component to the aqueous solution irradiated with the atmospheric pressure plasma.

A twelfth aspect of the present invention is drawn to a specific embodiment of the antitumor aqueous solution, wherein the aqueous solution is a culture solution, and the culture solution is irradiated with the atmospheric pressure plasma.

A thirteenth aspect of the present invention is drawn to a specific embodiment of the antitumor aqueous solution, which selectively kills cancer cells.

A fourteenth aspect of the present invention is drawn to a specific embodiment of the antitumor aqueous solution, which induces apoptosis of cancer cells by blocking at least one signal transduction pathway of AKT and ERK of the cancer cells.

A fifteenth aspect of the present invention is drawn to a specific embodiment of the antitumor aqueous solution, which kills cancer cells having resistance to an anticancer agent.

In a sixteenth aspect of the present invention, there is provided a method for producing an anticancer agent which kills cancer cells. The anticancer agent production method comprises an aqueous solution preparation step of preparing an aqueous solution through addition, to water, of a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O); and a plasma irradiation step of irradiating the aqueous solution with atmospheric pressure plasma generated in a plasma generation region by means of a plasma generator.

In a seventeenth aspect of the present invention, there is provided an anticancer agent which kills cancer cells. The anticancer agent is produced by dissolving, in water, a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O), to thereby prepare an aqueous solution, and irradiating the aqueous solution with atmospheric pressure plasma. The anticancer agent selectively kills cancer cells.

### Effects of the Invention

According to the present invention, there are provided an antitumor aqueous solution and an anticancer agent, both of which can kill cancer cells while having virtually no effects on normal cells, and methods for producing the antitumor aqueous solution and the anticancer agent.

### BRIEF DESCRIPTION OF THE DRAWINGS

FIG. 1 schematically illustrates the configuration of a robot arm which moves a gas ejection port of a plasma irradiation device.

FIG. 2.A is a cross-sectional view of the configuration of a first plasma irradiation device, and FIG. 2.B shows the shape of electrodes.

FIG. 3.A is a cross-sectional view of the configuration of a second plasma irradiation device, and FIG. 3.B is a partial cross-sectional view and shows a cross section perpendicular to the longitudinal direction of a plasma region.

FIG. 4 is a micrograph showing the results in the case of immersion of a cancer cell culture medium in a “plasma culture solution” in experiment A.

FIG. 5 is a micrograph showing the results in the case of immersion of a cancer cell culture medium in an “argon-gas-irradiated culture solution” in experiment A.

FIG. 6 is a micrograph showing the results in the case of immersion of a cancer cell culture medium in a “common culture solution” in experiment A.

FIG. 7 is a graph showing comparison of cancer cell viability in the cases of immersion of a cancer cell culture medium in a “common culture solution,” an “argon-gas-irradiated culture solution,” and a “plasma culture solution” in experiment B (number of cells: 1,000, plasma irradiation time: one minute).

FIG. 8 is a graph showing comparison of cancer cell viability in the cases of immersion of a cancer cell culture medium in a “common culture solution,” an “argon-gas-irradiated culture solution,” and a “plasma culture solution” in experiment B (number of cells: 5,000, plasma irradiation time: one minute).

FIG. 9 is a graph showing comparison of cancer cell viability in the cases of immersion of a cancer cell culture medium in a “common culture solution,” an “argon-gas-irradiated culture solution,” and a “plasma culture solution” in experiment B (number of cells: 10,000, plasma irradiation time: one minute).

FIG. 10 is a graph showing comparison of cancer cell viability in the cases of immersion of a cancer cell culture medium in a “common culture solution,” an “argon-gas-irradiated culture solution,” and a “plasma culture solution” in experiment B (number of cells: 1,000, plasma irradiation time: three minutes).

FIG. 11 is a graph showing comparison of cancer cell viability in the cases of immersion of a cancer cell culture medium in a “common culture solution,” an “argon-gas-irradiated culture solution,” and a “plasma culture solution” in experiment B (number of cells: 5,000, plasma irradiation time: three minutes).

FIG. 12 is a graph showing comparison of cancer cell viability in the cases of immersion of a cancer cell culture medium in a “common culture solution,” an “argon-gas-irradiated culture solution,” and a “plasma culture solution” in experiment B (number of cells: 10,000, plasma irradiation time: three minutes).

FIG. 13 is a graph showing comparison of cancer cell viability in the cases of immersion of a cancer cell culture medium in a “common culture solution,” an “argon-gas-irradiated culture solution,” and a “plasma culture solution” in experiment B (number of cells: 1,000, plasma irradiation time: five minutes).

FIG. 14 is a graph showing comparison of cancer cell viability in the cases of immersion of a cancer cell culture medium in a “common culture solution,” an “argon-gas-irradiated culture solution,” and a “plasma culture solution” in experiment B (number of cells: 5,000, plasma irradiation time: five minutes).

FIG. 15 is a graph showing comparison of cancer cell viability in the cases of immersion of a cancer cell culture medium in a “common culture solution,” an “argon-gas-irradiated culture solution,” and a “plasma culture solution” in experiment B (number of cells: 10,000, plasma irradiation time: five minutes).

FIG. 16 is a graph showing comparison between the effect of a “plasma culture solution” on cancer cells and that on normal cells in experiment C.

FIG. 17 is a graph showing the duration of the antitumor effect of a “plasma culture solution” in experiment D.

FIG. 18 shows the amount of expression of total AKT and the degree of activation of AKT, which is a signal transduction pathway of cells (experiment E).

FIG. 19 shows the amount of expression of total ERK and the degree of activation of ERK, which is a signal transduction pathway of cells (experiment E).

FIG. 20 shows the antitumor effect of a culture solution irradiated with argon-hydrogen plasma in experiment F.

FIG. 21 shows the selectivity of the antitumor effect of a culture solution irradiated with argon-hydrogen plasma in experiment F.

FIG. 22 is a graph showing the results of a test for examining the antitumor effect of a plasma solution in experiment G, the plasma solution being prepared by irradiating water with plasma, followed by addition of a culture solution.

FIG. 23 is a graph showing the results of a test for examining the antitumor effect of a plasma solution in experiment G, the plasma solution being prepared by irradiating an aqueous disodium hydrogen phosphate solution with plasma, followed by addition of a culture solution.

FIG. 24 is a graph showing the results of a test for examining the antitumor effect of a plasma solution in experiment G, the plasma solution being prepared by irradiating an aqueous sodium hydrogen carbonate solution with plasma, followed by addition of a culture solution.

FIG. 25 is a graph showing the results of a test for examining the antitumor effect of a plasma solution in experiment G, the plasma solution being prepared by irradiating an aqueous L-glutamine solution with plasma, followed by addition of a culture solution.

FIG. 26 is a graph showing the results of a test for examining the antitumor effect of a plasma solution in experiment G, the plasma solution being prepared by irradiating an aqueous L-histidine solution with plasma, followed by addition of a culture solution.

FIG. 27 is a graph showing the results of a test for examining the antitumor effect of a plasma solution in experiment G, the plasma solution being prepared by irradiating an aqueous L-tyrosine disodium dihydrate solution with plasma, followed by addition of a culture solution.

FIG. 28 is a graph showing the results of a test for examining the antitumor effect of plasma solutions in experiment G, the plasma solutions being prepared by irradiating various single-component aqueous solutions with plasma, followed by addition of a culture solution (part 1).

FIG. 29 is a graph showing the results of a test for examining the antitumor effect of plasma solutions in experiment G, the plasma solutions being prepared by irradiating various single-component aqueous solutions with plasma, followed by addition of a culture solution (part 2).

FIG. 30 is a graph showing the results of a test for examining the antitumor effect of plasma solutions in experiment G, the plasma solutions being prepared by irradiating various single-component aqueous solutions with plasma, followed by addition of a culture solution (part 3).

FIG. 31 is a graph showing the results of a test for examining the antitumor effect of plasma solutions in experiment G, the plasma solutions being prepared by irradiating various single-component aqueous solutions with plasma, followed by addition of a culture solution (part 4).

FIG. 32 is a graph showing the results of a test for examining the antitumor effect of a plasma solution in experiment G, the plasma solution being prepared by irradiating an aqueous solution containing five solutes with plasma, followed by addition of a culture solution.

FIG. 33 is a graph showing the results of a test for examining the concentration dependence of the antitumor effect of a plasma solution in experiment G, the plasma solution being prepared by irradiating an aqueous disodium hydrogen phosphate solution with plasma, followed by addition of a culture solution.

FIG. 34 is a graph showing the results of a test for examining the concentration dependence of the antitumor effect of a plasma solution in experiment G, the plasma solution being prepared by irradiating an aqueous sodium hydrogen carbonate solution with plasma, followed by addition of a culture solution.

FIG. 35 is a graph showing the results of a test for examining the antitumor effect of a plasma solution in experiment G, the plasma solution being prepared by irradiating an aqueous potassium chloride solution with plasma, followed by addition of a culture solution.

FIG. 36 is a graph showing the results of a test for examining the antitumor effect of a plasma solution in experiment G, the plasma solution being prepared by irradiating an aqueous sodium chloride solution with plasma, followed by addition of a culture solution.

FIG. 37 is a graph showing the results of a test for examining the antitumor effect of a plasma culture solution on ovarian cancer cells having resistance to an anticancer agent in experiment H (part 1).

FIG. 38 is a graph showing the results of a test for examining the antitumor effect of a plasma culture solution on ovarian cancer cells having resistance to an anticancer agent in experiment H (part 2).

FIG. 39 is a micrograph showing the case of administration of a common culture solution or a plasma culture solution to cells having or not having resistance to an anticancer agent in experiment H (part 1).

FIG. 40 is a micrograph showing the case of administration of a common culture solution or a plasma culture solution to cells having or not having resistance to an anticancer agent in experiment H (part 2).

FIG. 41 is a micrograph showing the case of administration of a common culture solution or a plasma culture solution to cells having or not having resistance to an anticancer agent in experiment H (part 3).

FIG. 42 is a micrograph showing the case of administration of a common culture solution or a plasma culture solution to cells having or not having resistance to an anticancer agent in experiment H (part 4).

FIG. 43 is a micrograph showing the case of administration of a common culture solution or a plasma culture solution to cells having or not having resistance to an anticancer agent in experiment H (part 5).

FIG. 44 is a micrograph showing the case of administration of a common culture solution or a plasma culture solution to cells having or not having resistance to an anticancer agent in experiment H (part 6).

FIG. 45 is a photograph showing comparison of the results of administration of a plasma culture solution and a common culture solution to nude mice inoculated with ovarian cancer cells in experiment I (part 1).

FIG. 46 is a photograph showing comparison of the results of administration of a plasma culture solution and a common culture solution to nude mice inoculated with ovarian cancer cells in experiment I (part 2).

FIG. 47 is a graph showing a change in tumor volume in the case of administration of a plasma culture solution or a common culture solution to nude mice inoculated with ovarian cancer cells in experiment I (part 1).

FIG. 48 is a graph showing a change in tumor volume in the case of administration of a plasma culture solution or a common culture solution to nude mice inoculated with ovarian cancer cells in experiment I (part 1).

FIG. 49 is a graph showing the weight of tumor in nude mice inoculated with ovarian cancer cells 28 days after administration of a plasma culture solution or a common culture solution to the nude mice in experiment I.

### MODES FOR CARRYING OUT THE INVENTION

Specific embodiments will next be described with reference to the drawings by taking, as examples, a plasma solution and a production method therefor.

### 1. PLASMA SOLUTION PRODUCTION APPARATUS

### 1-1. Configuration of Plasma Solution Production Apparatus

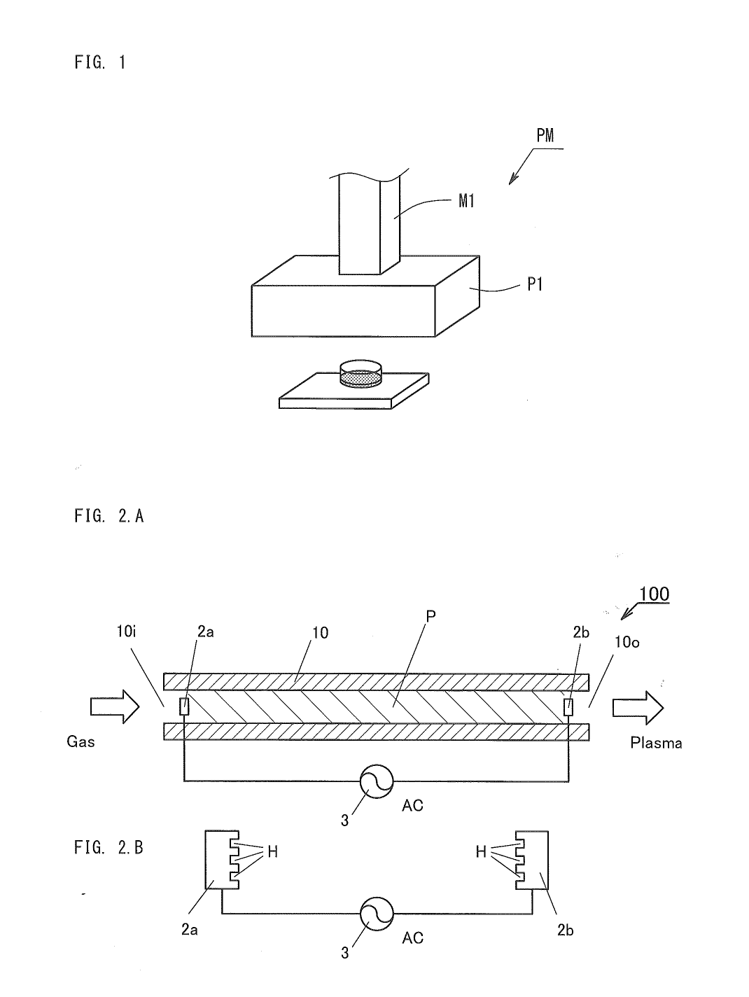

As shown in FIG. 1, the plasma solution production apparatus PM of the present embodiment includes a plasma irradiation device P1 and an arm robot M1. The plasma irradiation device P1 is employed for generating plasma, and applying the plasma to a solution. As described hereinbelow, the plasma irradiation device P1 has two types (i.e., a first plasma irradiation device 100 and a second plasma irradiation device 200). Any of these types may be employed.

As shown in FIG. 1, the arm robot M1 can move the plasma irradiation device P1 in x-axis, y-axis, and z-axis directions. For the sake of convenience of description, the direction of plasma irradiation corresponds to a -z-axis direction. The arm robot M1 can adjust the distance between the level of a solution and the plasma irradiation device P1. The plasma solution production apparatus PM can apply plasma for a predetermined plasma irradiation time.

### 1-2. First Plasma Irradiation Device

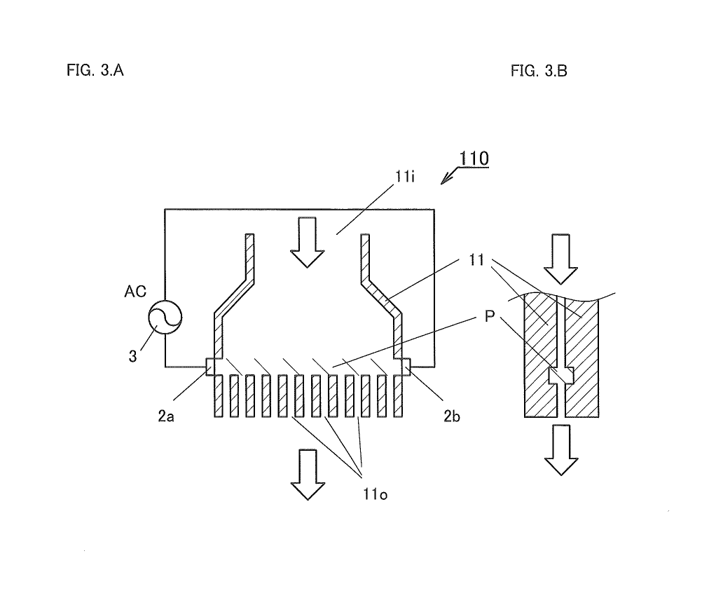

FIG. 2.A is a schematic cross-sectional view of the configuration of a plasma irradiation device 100. The plasma irradiation device 100 corresponds to a first plasma irradiation device which ejects plasma in a pointwise manner. FIG. 2.B details the shape of electrodes 2a and 2b of the plasma irradiation device 100 shown in FIG. 2.A.

The plasma irradiation device 100 includes a housing 10, electrodes 2a and 2b, and a voltage application unit 3. The housing 10 is formed of sintered alumina (Al₂O₃). The housing 10 has a tubular shape. The housing 10 has an inner diameter of 2 to 3 mm. The housing 10 has a thickness of 0.2 to 0.3 mm. The housing 10 has a length of 25 cm. The housing 10 has, at opposite ends thereof, a gas inlet port 10i and a gas ejection port 10o. A gas for generating plasma is introduced through the gas inlet port 10i. Plasma is ejected through the gas ejection port 10o to the outside of the housing 10. The direction of flow of a gas is shown by arrows in FIG. 2.A.

The paired electrodes 2a and 2b are located so as to face each other. The length (in a facing direction) of each of the electrodes 2a and 2b is smaller than the inner diameter of the housing 10, and is, for example, about 1 mm. As shown in FIG. 2.B, each of the electrodes 2a and 2b has numerous hollows H on its facing surface. That is, the facing surface of each of the electrodes 2a and 2b is finely embossed. Each hollow H has a depth of about 0.5 mm.

The electrode 2a is provided inside of the housing 10 and in the vicinity of the gas inlet port 10i. The electrode 2b is provided inside of the housing 10 and in the vicinity of the gas ejection port 10o. Therefore, in the plasma irradiation device 100, a gas is introduced from the side opposite the facing surface of the electrode 2a, and is ejected to the side opposite the facing surface of the electrode 2b. The distance between the electrodes 2a and 2b is 24 cm. The distance between the electrodes 2a and 2b may be smaller than 24 cm.

The voltage application unit 3 applies AC voltage between the electrodes 2a and 2b. The voltage application unit 3 increases commercial AC voltage (60 Hz, 100 V) to 9 kV and applies the voltage between the electrodes 2a and 2b.

When voltage is applied between the electrodes 2a and 2b by means of the voltage application unit 3 while argon is introduced through the gas inlet port 10i, plasma is generated in the interior of the housing 10. As shown by diagonal lines in FIG. 2.A, the plasma generation region is represented by P. The plasma generation region P is covered with the housing 10.

### 1-3. Second Plasma Irradiation Device.

FIG. 3.A is a schematic cross-sectional view of the configuration of a plasma irradiation device 110. The plasma irradiation device 110 corresponds to a second plasma irradiation device which ejects plasma in a linear manner. FIG. 3.B is a partial cross-sectional view of the plasma irradiation device 110 shown in FIG. 3.A, and shows a cross section perpendicular to the longitudinal direction of a plasma region P.

The plasma irradiation device 110 includes a housing 11, electrodes 2a and 2b, and a voltage application unit 3. The housing 11 is formed of sintered alumina (Al₂O₃). The housing 11 has, at opposite ends thereof, a gas inlet port 11i and numerous gas ejection ports 11o. The gas inlet port 11i, whose longitudinal direction corresponds to the horizontal direction of FIG. 3.A, assumes a slit-like shape. The width of the slit extending from the gas inlet port 11i to a portion directly above the plasma region P (i.e., the width in the horizontal direction of FIG. 3.B) is 1 mm.

Plasma is ejected through the gas ejection ports 11o to the outside of the housing 11. Each of the gas ejection ports 11o has a cylindrical or slit-like shape. When the gas ejection ports 11o have a cylindrical shape, they are arranged linearly in the longitudinal direction of the plasma region. Each of the gas ejection ports 11o has an inner diameter of 1 to 2 mm. When the gas ejection ports 11o have a slit-like shape, the slit width of each gas ejection port 110 is preferably 1 mm or less. In such a case, stable plasma is generated. The gas inlet port 11i is provided so as to introduce a gas in a direction crossing with a line connecting the electrode 2a and the electrode 2b.

The electrodes 2a and 2b and the voltage application unit 3 are the same as those of the plasma irradiation device 100 shown in FIG. 1. Similar to the case of the plasma irradiation device 100, commercial AC voltage is increased and applied between the electrodes 2a and 2b. Thus, plasma can be ejected in a linear manner.

When a plurality of plasma irradiation devices 110, each of which ejects plasma in a linear manner, are aligned in the horizontal direction of FIG. 3.B, plasma can be ejected in a certain rectangular planar region.

In experiments described hereinbelow, there was employed a plasma irradiation device having a plurality of gas ejection ports 110 and capable of ejecting plasma in a generally circular planar region.

### 2. PLASMA GENERATED BY PLASMA IRRADIATION DEVICE

The plasma generated by means of the plasma irradiation device 100 or 110 is non-equilibrium atmospheric pressure plasma. As used herein, the term “atmospheric pressure plasma” refers to plasma having a pressure of 0.5 atm to 2.0 atm.

In the present embodiment, Ar gas is generally employed as a plasma-generating gas. Needless to say, electrons and Ar ions are generated in the plasma generated by means of the plasma irradiation device 100 or 110. The Ar ions generate UV rays. Since the plasma is released in air, oxygen radicals or nitrogen radicals are generated.

The plasma has a density of 1×10¹⁴ cm⁻³ to 1×10¹⁷ cm⁻³. Plasma generated through dielectric barrier discharge has a density of about 1×10¹¹ cm⁻³ to about 1×10¹³ cm⁻³. That is, the density of the plasma generated by means of the plasma irradiation device 100 or 110 is about 1,000 times that of the plasma generated through dielectric barrier discharge. Therefore, a larger amount of Ar ions are generated in the plasma generated by means of the plasma irradiation device, and thus large amounts of radicals or UV rays are generated. The plasma density is almost equal to the density of electrons in the plasma.

The plasma temperature during generation of the plasma is about 1,000 K to about 2,500 K. The electron temperature of the plasma is higher than the gas temperature. Furthermore, even when the electron density is 1×10¹⁴ cm⁻³ to 1×10¹⁷ cm⁻³, the gas temperature is about 1,000 K to about 2,500 K. The plasma temperature corresponds to the temperature as measured in the plasma generation region P. Therefore, the plasma temperature at cancer cells can be adjusted to room temperature or thereabouts by varying plasma conditions or the distance between the gas ejection port and the cancer cells. Thus, when the plasma is applied to the cancer cells and normal cells, there is virtually no heat damage to these cells.

The oxygen radical density is 2×10¹⁴ cm⁻³ to 1.6×10¹⁵ cm⁻³. The oxygen radical density can be adjusted by regulating the amount of oxygen gas incorporated into the argon gas employed.

### 3. PLASMA SOLUTION

The plasma solution of the present embodiment is produced by irradiating a raw material solution with plasma for a predetermined period of time. As used herein, the term “raw material solution” refers to an aqueous solution prepared from an aqueous solvent. The raw material solution employed is prepared by mixing water with a culture component. That is, the raw material solution corresponds to a culture solution for culturing of, for example, cells. The culture solution may be, for example, DMEM. DMEM contains a sugar such as glucose. As used herein, the term “culture component” refers to a component contained in a culture solution for culturing of, for example, cells. The culture component may be, for example, one described below in both Table 3 (DMEM components) and Table 9 (RPMI 1640 components).

### 4. PLASMA SOLUTION PRODUCTION METHOD

The plasma solution of the present embodiment may be produced through any of two methods. These two methods will next be described.

### 4-1. Plasma Solution Production Method (First Method)

### 4-1-1. Aqueous Solution Preparation Step (First Method)

The first method will now be described. There is prepared, as an aqueous solution, a culture solution containing components shown below in Table 3 (DMEM components) or in Table 9 (RPMI 1640 components). That is, there is provided a culture solution prepared by adding these culture components to water.

### 4-1-2. Plasma Irradiation Step (First Method)

Next, the culture solution is irradiated with atmospheric pressure plasma generated in the plasma generation region by means of the aforementioned plasma generator. During the course of plasma irradiation, the distance between the level of the solution and the plasma ejection port is adjusted to, for example, 1 cm. The distance may be varied to fall within a range of 0.5 cm to 3 cm. The density of the plasma is 1×10¹⁴ cm⁻³ to 1×10¹⁷ cm⁻³. The plasma temperature is about 1,000 K to about 2,500 K. The plasma temperature may be lowered to room temperature or thereabouts (about 300 K) at the level of the solution. The oxygen radical density is 2×10¹⁴ cm⁻³ to 1.6×10¹⁵ cm⁻³. These plasma conditions are summarized in Table 1.

TABLE 1

| Conditions | Numerical range |
|---|---|
| Distance between solution level and | 0.5 cm to 3 cm |
| ejection port | |
| Plasma density | 1 × 10¹⁴ cm⁻³ to 1 × 10¹⁷ cm⁻³ |
| Plasma temperature | 1000 K to 2500 K |
| Oxygen radical density | 2 × 10¹⁴ cm⁻³ to 1.6 × 10¹⁵ cm⁻³ |

As described hereinbelow in experiments, in order to produce a plasma solution exhibiting antitumor effect, the plasma density-time product is adjusted to satisfy the following: 1.2×10¹⁸ sec·cm⁻³ or more. As used herein, the “plasma density-time product” is defined by the product of the plasma density in the plasma generation region and the time during which the aqueous solution is irradiated with the atmospheric pressure plasma (irradiation time).

### 4-2. Plasma Solution Production Method (Second Method)

### 4-2-1. Aqueous Solution Preparation Step (First Method)

There is prepared an aqueous solution through addition, to water, of a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O).

### 4-2-2. Plasma Irradiation Step (Second Method)

Next, the aqueous solution is irradiated with atmospheric pressure plasma generated in the plasma generation region by means of the aforementioned plasma generator. Conditions for plasma irradiation are the same as those employed in the first method.

### 4-2-3. Culture Component Addition Step (Second Method)

Subsequently, components shown below in Table 3 (DMEM components) or in Table 9 (RPMI 1640 components) are added to the aqueous solution which has been irradiated with the atmospheric pressure plasma.

### 5. CANCER TREATMENT WITH PLASMA SOLUTION

### 5-1. Property and Application of Plasma Solution (Anticancer Agent)

As shown hereinbelow in the experimental results, the plasma solution is an antitumor aqueous solution which kills cancer cells (i.e., the solution exhibits antitumor effect). That is, the plasma solution also serves as an anticancer agent exhibiting anticancer effect. This anticancer effect is exerted until the lapse of less than 18 hours from initiation of plasma irradiation. A plasma solution which has been irradiated with the plasma for one minute or more exhibits anticancer effect. As described below, the plasma solution of the present embodiment causes virtually no damage to normal cells.

### 6. EXPERIMENT A

### Antitumor Effect of Plasma Culture Solution and Immersion Time

This experiment was carried out for examining the antitumor effect of a plasma culture solution. This experiment was also carried out for determining the relationship between killing of cancer cells and the time during which the cancer cells are exposed to the plasma culture solution. The plasma culture solution corresponds to a culture solution irradiated with plasma; i.e., a type of plasma solution.

### 6-1. Cancer Cells Employed

Glioma cells were employed in this experiment. Glioma occurs in neuroglial cells (glial cells); i.e., glioma is a type of brain tumor. There were employed glioma cells shown in Table 2; specifically, U251SP cells and U87MG cells.

TABLE 2

| Cell name | State | Type |
|---|---|---|
| U251SP | Cancer cells | Glioma cells |
| U87MG | Cancer cells | Glioma cells |
| RI-371 | Normal cells | Astrocytes |

### 6-2. Experimental Method

### 6-2-1. Cancer Cell Culture Medium

A cancer cell culture medium was prepared through culturing of the aforementioned cancer cells in a plate (i.e., a plastic-made container). Then, a culture solution was added into the plate. The culture solution was prepared by mixing DMEM, serum (FBS), and antibiotics (penicillin and streptomycin). Components of DMEM are shown in Table 3.

TABLE 3

Calcium chloride
Ferric nitrate•9H₂O
Magnesium sulfate (anhydrous)
Potassium chloride
Sodium hydrogen carbonate
Sodium chloride
Monosodium phosphate (anhydrous)
L-Arginine•HCl
L-Cystine•2HCl
L-Glutamine
Glycine
L-Histidine•HCl•H₂O
L-Isoleucine
L-Leucine
L-Lysine•HCl
L-Methionine
L-Phenylalanine
L-Serine
L-Threonine
L-Tryptophan
L-Tyrosine•2Na•2H₂O
L-Valine
Choline chloride
Folic acid
myo-Inositol
Niacinamide
D-Pantothenic acid
Pyridoxine•HCl
Riboflavin
Thiamine•HCl
D-Glucose
Phenol red•Na

### 6-2-2. Preparation of Plasma Culture Solution

A plasma culture solution was prepared separately to the preparation of the cancer cell culture medium. In this experiment, a plate having six holes was employed. These holes are non-through holes. Therefore, the solution can be added into each hole. Firstly, the culture solution (3 mL) is added into each hole of the plate. The culture solution employed was prepared in the aforementioned manner by mixing DMEM, serum (FBS), and antibiotics (penicillin and streptomycin). Components of DMEM are shown in Table 3.

Subsequently, the culture solution is irradiated with plasma by means of the plasma solution production apparatus PM. In this case, the aqueous solution was irradiated with atmospheric pressure plasma while the level of the culture solution was adjusted so that the culture solution was not exposed to the plasma generation region. Then, the aqueous solution was irradiated with the atmospheric pressure plasma while the facing electrodes of the plasma solution production apparatus PM were located outside the culture solution so that the culture solution was not provided between the electrodes. Thus, although the culture solution is not exposed to the plasma generation region, the culture solution is irradiated with various radicals generated in the plasma. In this case, the culture solution contained in the plate is irradiated with the plasma so that the plasma pushes out air above the culture solution. Therefore, during the course of plasma irradiation, the culture solution is barely exposed to air.

Table 4 shows plasma irradiation conditions. Only argon gas was employed for generating plasma. The gas flow rate was adjusted to 2.0 slm. The distance between the plasma ejection port and the solution level was adjusted to 13 mm. The plasma irradiation time was adjusted to five minutes. The plasma density in the plasma generation region was found to be 2×10¹⁶ cm⁻³.

TABLE 4

| | | |
|---|---|---|
| Gas flow rate | 2.0 | slm |
| Distance between plasma ejection port and | 13 | mm |
| solution level | | |
| Plasma irradiation time | 5 | minutes |
| Plasma density (at the time of generation) | 2 × 10¹⁶ | cm⁻³ |

### 6-2-3. Supply of Plasma Culture Solution to Cancer Cell Culture Medium

Subsequently, the plasma culture solution is supplied to the cancer cell culture medium. Specifically, the culture solution is removed from the cancer cell culture medium, and the plasma culture solution is added to the cancer cell culture medium. In this case, the amount of the plasma culture solution supplied is 0.2 mL. After the lapse of a predetermined period of time following exchange of the culture solution with the plasma culture solution, the culture solution is exchanged again. The culture solution supplied to the cancer cell culture medium is a common culture solution.

Cancer cell viability was examined by varying the time during which cancer cells were immersed in the plasma culture solution. Cancer cell viability was examined 16 hours after supply of the plasma culture solution to the cancer cell culture medium. In this case, the number of surviving cancer cells was counted through microscopic observation.

### 6-3. Experimental Results

Table 5 shows the experimental results. In Table 5, numerical values shown below the cancer cell strains (U251SP and U87MG) correspond to cancer cell viability. The numerical value “1” corresponds to survival of cancer cells, whereas the numerical value “0” corresponds to killing of all cancer cells. Meanwhile, the numerical value “0.6” corresponds to the case where the ratio of the number of surviving cancer cells to that of cancer cells before supply of the plasma culture solution is about 60%.

As shown in Table 5, when the immersion time is 30 minutes or longer, cancer cells are killed. That is, killing of cancer cells requires immersion of the cancer cells in the plasma culture solution for 30 minutes or longer. When the immersion time is 30 minutes or longer and shorter than 60 minutes, the plasma culture solution exhibits antitumor effect.

In Table 5, “Untreated” corresponds to the case where cancer cells were treated not with the plasma culture solution but with a common culture solution, and “Ar gas” corresponds to the case where the culture solution was irradiated not with the plasma but with only Ar gas. These cases correspond to comparative examples for indicating that the plasma culture solution has the effect of killing cancer cells.

TABLE 5

| | | |
|---|---|---|
| Immersion time | U251SP | U87MG |

| | | | |
|---|---|---|---|
| 1 | minute | 1 | 1 |
| 5 | minutes | 1 | 1 |
| 10 | minutes | 1 | 1 |
| 30 | minutes | 1 | 1 |
| 60 | minutes | 0.1 | 0.6 |
| 120 | minutes | 0 | 0 |
| 16 | hours | 0 | 0 |

| | | |
|---|---|---|
| Untreated | 1 | 1 |
| Ar gas | 1 | 1 |

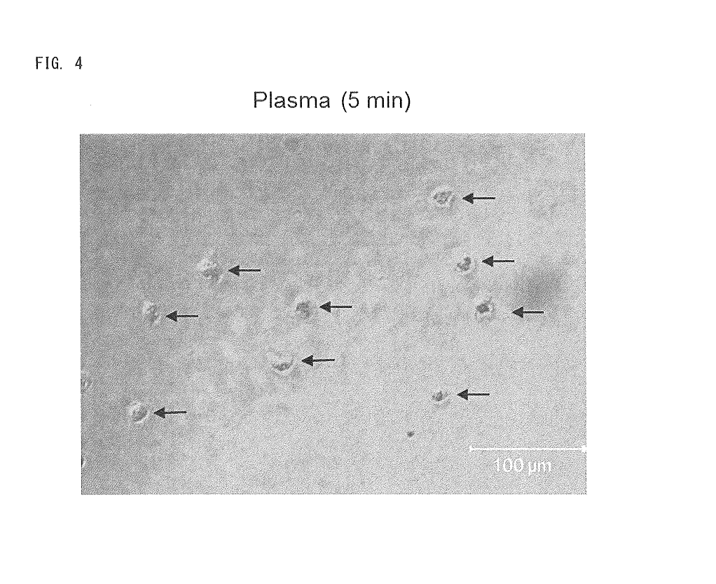

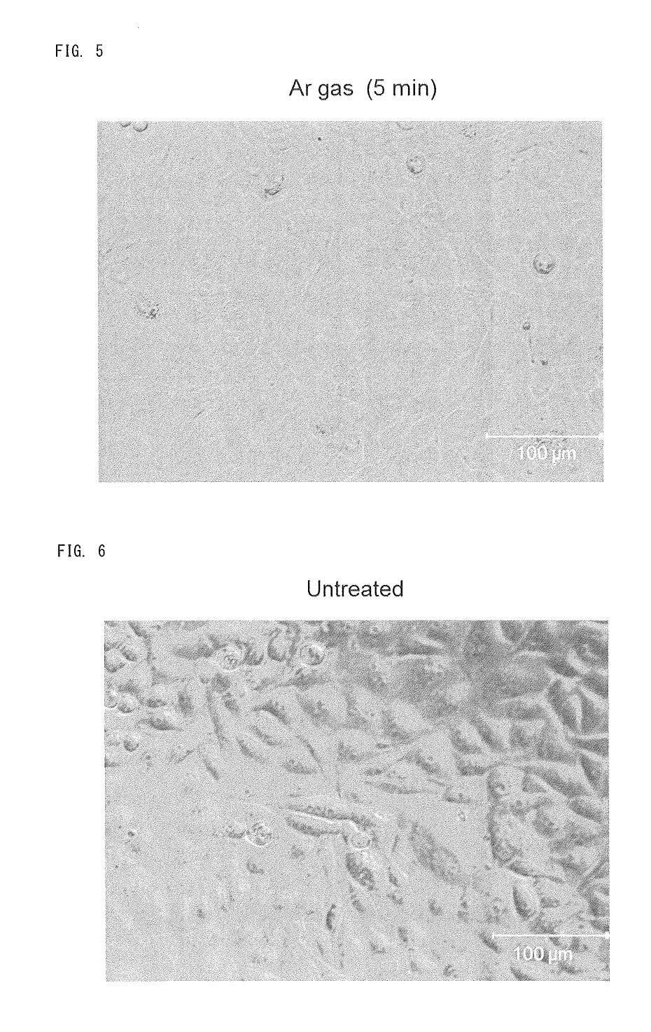

FIGS. 4 to 6 show actual micrographs. All of these cancer cells are U251SP cells. FIG. 4 is a micrograph showing the case where cancer cells were immersed in the plasma culture solution. FIG. 4 corresponds to the case of “16 hours” shown in Table 5. FIG. 5 is a micrograph showing the case where cancer cells were immersed in the culture solution irradiated with argon gas. FIG. 5 corresponds to the case of “Ar gas” shown in Table 5. FIG. 6 is a micrograph showing the case where the plasma culture solution was exchanged with a common culture solution. FIG. 6 corresponds to the case of “Untreated” shown in Table 5.

The bar shown in each of FIGS. 4 to 6 corresponds to a length of 100 μm. In FIG. 4, cancer cells killed through apoptosis induction are shown by arrows.

Thus, the plasma culture solution exhibits antitumor effect. That is, the plasma culture solution serves as an anticancer agent exhibiting antitumor effect.

### 7. EXPERIMENT B

### Plasma Irradiation Time and Antitumor Effect

### 7-1. Cancer Cells Employed

In this experiment, U251SP cells (glioma cells) shown in Table 2 were employed as cancer cells.

### 7-2. Experimental Method

In this experiment, a cancer cell culture medium was immersed in a plasma culture solution in the same manner as in experiment A. In this experiment, antitumor effect was examined by employing combinations of plasma culture solutions and cancer cell culture media. Three plasma culture solutions (corresponding to the following different plasma irradiation times) were prepared.

Plasma irradiation time: 1 minute

Plasma irradiation time: 3 minutes

Plasma irradiation time: 5 minutes

Cancer cell culture media containing different numbers of cancer cells (i.e., having different cancer cell densities) were prepared. Specifically, three cancer cell culture media corresponding to the following cell numbers were prepared.

Cancer cells (U251SP): 1,000 cells

Cancer cells (U251SP): 5,000 cells

Cancer cells (U251SP): 10,000 cells

The experiment was carried out on nine samples of “plasma culture solution” (immersion of cancer cells) prepared from combinations of three different plasma irradiation times and three different numbers of cancer cells. For comparison, the experiment was also carried out on nine samples of “argon-gas-irradiated culture solution” (immersion of cancer cells). For another comparison, the experiment was also carried out on three samples of a common culture solution (immersion of cancer cells) with different numbers of cancer cells. That is, the experiment was carried out on these 21 samples.

### 7-3. Experimental Results

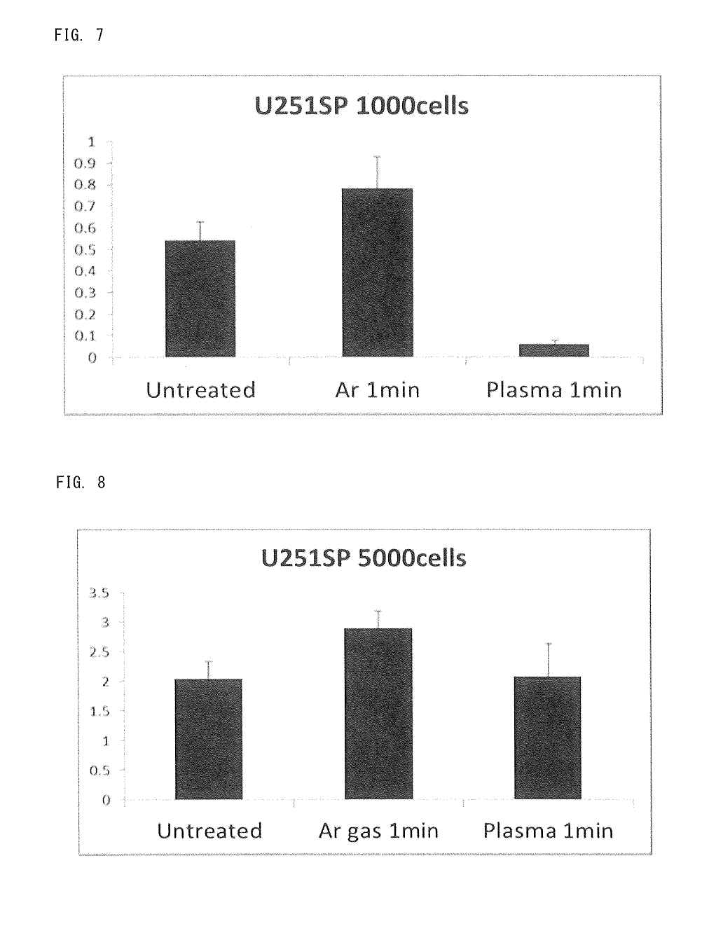

FIGS. 7 to 15 shows the experimental results. In each of these figures, the vertical axis corresponds to the number of cancer cells (arbitrary unit); specifically, 1,000 cancer cells correspond to about 0.5, 5,000 cancer cells correspond to about 2, and 10,000 cancer cells correspond to about 4.

### 7-3-1. One-Minute Irradiation

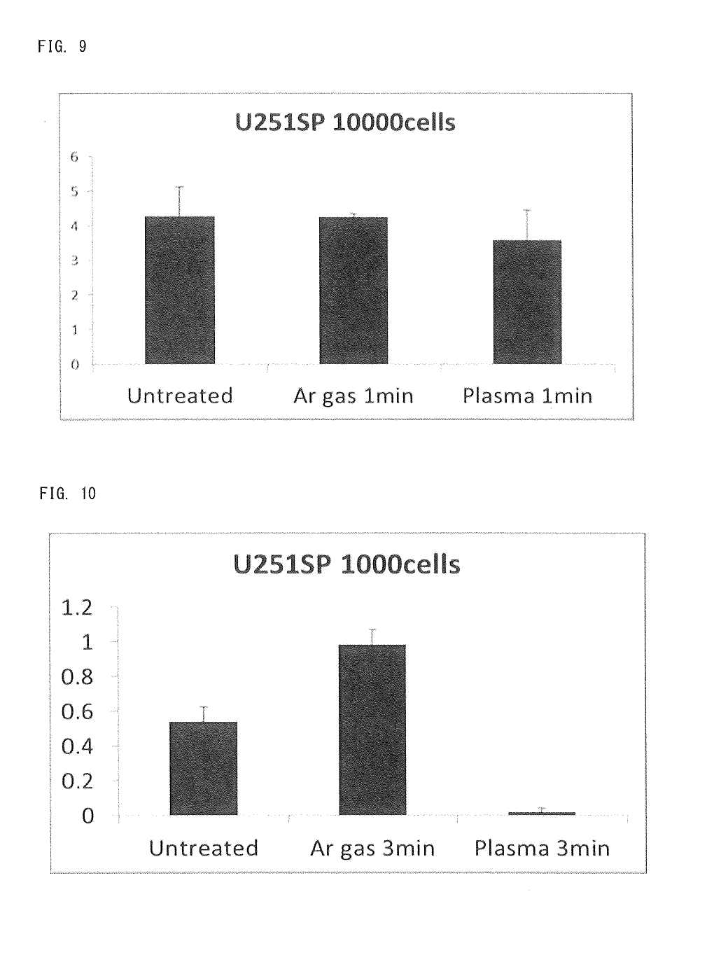

FIG. 7 shows the case where 1,000 cancer cells were immersed in a plasma culture solution (plasma irradiation time: one minute). FIG. 8 shows the case where 5,000 cancer cells were immersed in a plasma culture solution (plasma irradiation time: one minute). FIG. 9 shows the case where 10,000 cancer cells were immersed in a plasma culture solution (plasma irradiation time: one minute). In the case shown in FIG. 7 (one minute, 1,000 cells), the plasma culture solution exhibited antitumor effect. In contrast, in the cases shown in FIG. 8 (one minute, 5,000 cells) and FIG. 9 (one minute, 10,000 cells), no antitumor effect was observed.

### 7-3-2. Three-Minute Irradiation

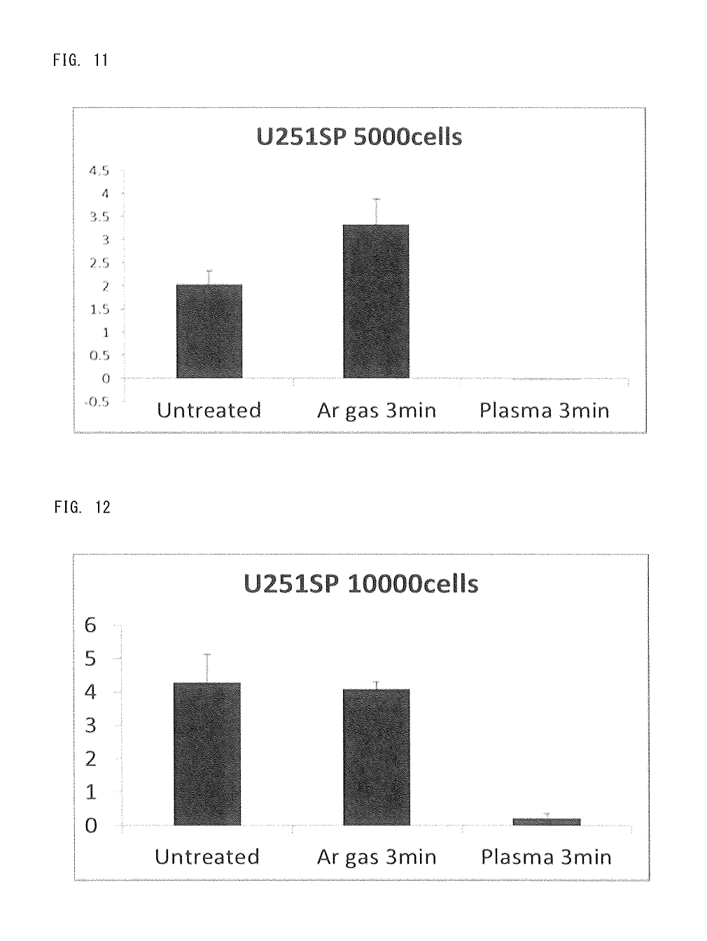

FIG. 10 shows the case where 1,000 cancer cells were immersed in a plasma culture solution (plasma irradiation time: three minutes). FIG. 11 shows the case where 5,000 cancer cells were immersed in a plasma culture solution (plasma irradiation time: three minutes). FIG. 12 shows the case where 10,000 cancer cells were immersed in a plasma culture solution (plasma irradiation time: three minutes). In all the cases shown in FIGS. 10 to 12, antitumor effect was observed.

### 7-3-3. Five-Minute Irradiation

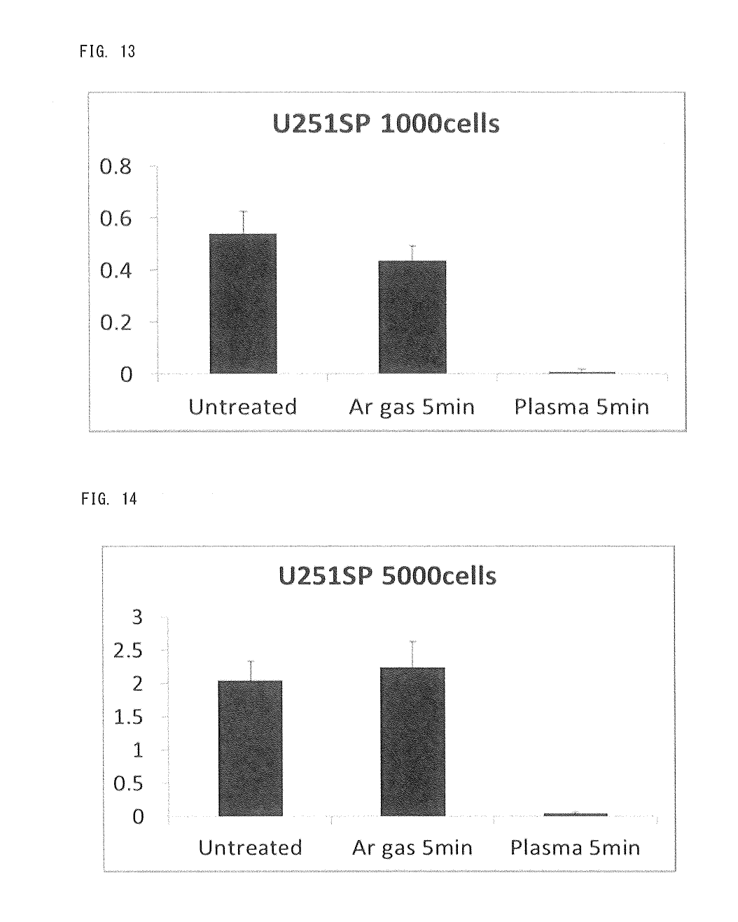

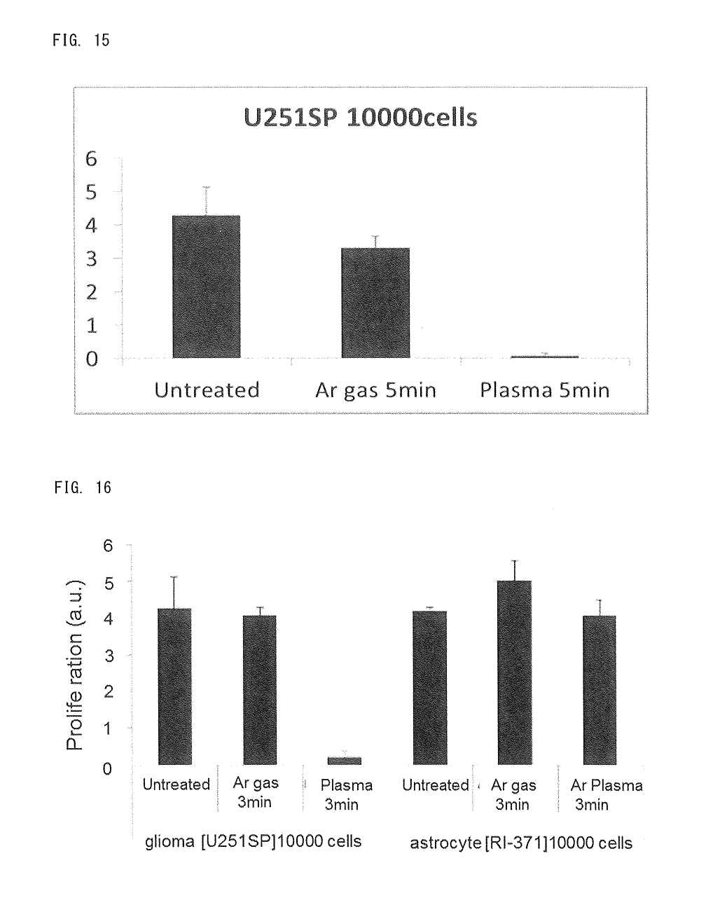

FIG. 13 shows the case where 1,000 cancer cells were immersed in a plasma culture solution (plasma irradiation time: five minutes). FIG. 14 shows the case where 5,000 cancer cells were immersed in a plasma culture solution (plasma irradiation time: five minutes). FIG. 15 shows the case where 10,000 cancer cells were immersed in a plasma culture solution (plasma irradiation time: five minutes). In all the cases shown in FIGS. 13 to 15, antitumor effect was observed.

Thus, when a culture solution is irradiated with atmospheric pressure plasma (plasma density: 2×10¹⁶ cm⁻³) for 60 seconds or longer, the resultant plasma culture solution exhibits antitumor effect. That is, a plasma density-time product of 1.2×10¹⁸ sec·cm⁻³ or more is preferred.

The larger the number of cancer cells, the higher the cancer cell viability. This indicates that a substance exhibiting antitumor effect is generated in the plasma culture solution, and the substance affects cancer cells and is consumed by them. Therefore, a plasma density-time product of 3.6×10¹⁸ sec·cm⁻³ or more is more preferred. This plasma density-time product corresponds to the case where a culture solution is irradiated with atmospheric pressure plasma (plasma density: 2×10¹⁶ cm⁻³) for 180 seconds or longer.

### 8. EXPERIMENT C

### Comparison Between Effect on Cancer Cells and Effect on Normal Cells

### 8-1. Cancer Cells and Normal Cells Employed

In this experiment, U251SP cells (glioma cells) shown in Table 2 were employed as cancer cells, and RI-371 cells (astrocytes) shown in Table 2 were employed as normal cells, for comparing the effect of a plasma culture solution on cancer cells with the effect thereof on normal cells.

### 8-2. Experimental Method

A plasma culture solution was supplied to a cancer cell culture medium and a normal cell culture medium. This experiment was carried out in the same manner as in the aforementioned experiment A. In this experiment, the number of cells was adjusted to 10,000 in each culture medium. The culture medium was immersed in the plasma culture solution.

### 8-3. Experimental Results

FIG. 16 shows the experimental results. As shown in FIG. 16, cancer cells (glioma cells: U251SP) are killed through immersion in the plasma culture solution. In contrast, virtually no normal cells (astrocytes: RI-371) are killed through immersion in the plasma culture solution. The number of normal cells immersed in the plasma culture solution is almost equal to that of normal cells immersed in a common culture solution. These data indicate that the plasma culture solution kills cancer cell, but barely kills normal cells. Thus, the plasma culture solution can selectively kill cancer cells. That is, the plasma culture solution can be employed for treatment of brain tumor.

### 9. EXPERIMENT D

### Duration of Antitumor Effect

Now will be described an experiment carried out on the duration of the antitumor effect of a plasma culture solution.

### 9-1. Cancer Cells Employed

In this experiment, U251SP cells (glioma cells) shown in Table 2 were employed as cancer cells.

### 9-2. Experimental Method

A cancer cell culture medium was immersed in a plasma culture solution in the same manner as described in experiment A. In this experiment, plasma culture solutions with different elapsed times from plasma irradiation were prepared, and the antitumor effect of each of the plasma culture solutions was examined. The plasma culture solutions prepared correspond to culture solutions which were irradiated with plasma for one minute and then allowed to stand for 0 hours, 1 hour, 8 hours, and 18 hours.

### 9-3. Experimental Results

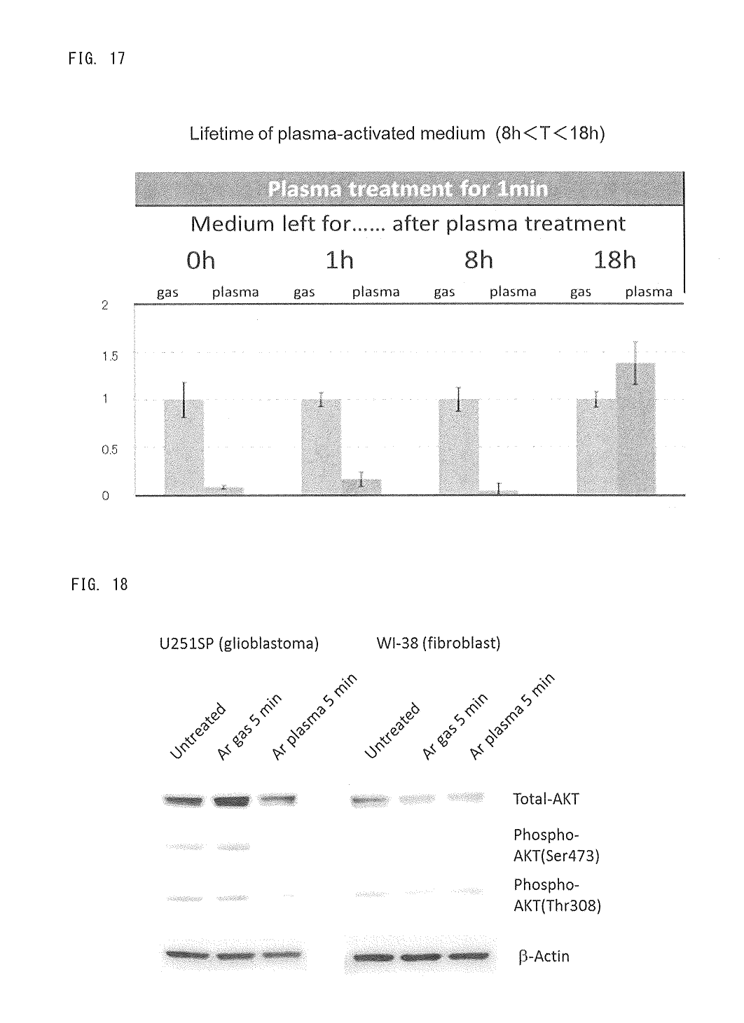

FIG. 17 shows the experimental results. As shown in FIG. 17, the plasma culture solution sustains its antitumor effect for at least eight hours from immediately after plasma irradiation. The antitumor effect of the plasma culture solution is lost before the lapse of 18 hours from plasma irradiation. That is, the plasma culture solution sustains its antitumor effect until the lapse of less than 18 hours from initiation of plasma irradiation.

### 10. EXPERIMENT E

### Signal Transduction Pathway

### 10-1. Cells Employed

In this experiment, U251SP cells (glioma cells) shown in Table 6 were employed as cancer cells, and WI-38 cells (fibroblasts) shown in Table 6 were employed as normal cells.

TABLE 6

| | |
|---|---|
| U251SP | Glioma cells (cancer cells) |
| WI-38 | Fibroblasts (normal cells) |

### 10-2. Solution Employed

In this experiment, three solutions were employed as shown in Table 7. Solution 1 is an untreated culture solution. Solution 2 is a culture solution irradiated with argon gas for five minutes. Solution 3 is a culture solution irradiated with argon plasma for five minutes. The culture component employed in this experiment is DMEM as in the case of experiment A. However, in this experiment, DMEM is not mixed with serum (FBS) and antibiotics (penicillin and streptomycin).

TABLE 7

| Name | Culture component | Irradiation |
|---|---|---|
| Solution 1 | DMEM | Untreated |
| Solution 2 | DMEM | Ar gas irradiation |
| | | (irradiation time: 5 minutes) |
| Solution 3 | DMEM | Ar plasma irradiation |
| | | (irradiation time: 5 minutes) |

### 10-3. Experimental Method

### 10-3-1. Preparation of Sample

The aforementioned U251SP cells (glioma cells) and WI-38 cells (fibroblasts) were inoculated into a plate (6-well plate). These cells were cultured in a common culture medium (DMEM) in the plate for 24 hours. Culture solutions 1, 2, and 3 were added to the U251SP cells (glioma cells) and the WI-38 cells (fibroblasts), and these cells were cultured in culture solutions 1, 2, and 3 for four hours, to thereby prepare six samples.

### 10-3-2. Western Blotting

The thus-prepared six samples were lysed in a RIPA cell lysis solution, to thereby prepare six cell lysates. These six cell lysates are fixed to a membrane through western blotting. Specifically, the cell lysates are subjected to electrophoresis, and the thus-separated cells are transferred to a membrane and then fixed to the membrane.

Thereafter, the degree of activation of signal transduction pathways was determined in the respective cells. Specifically, two signal transduction pathways of AKT and ERK were assayed. Regarding AKT, the degree of activation of Phospho-AKT (Ser473) or Phospho-AKT (Thr308) was determined, and the total amount of AKT (Total-AKT) was also determined.

Regarding ERK, the degree of activation of Phospho-ERK1 (Thr202/Tyr204) was determined. As used herein, the term “activation” refers to phosphorylation of AKT or ERK. Activation of AKT requires phosphorylation of two sites of Ser473 and Thr308.

### 10-4. Experimental Results

### 10-4-1. AKT

FIG. 18 shows the degree of activation of AKT. No activation of AKT was observed only in the case of U251SP cells (glioma cells) to which culture solution 3 (i.e., solution irradiated with argon plasma) was added. However, slight antibody response was observed at Phospho-AKT (Thr308). In contrast, in U251SP cells (glioma cells) to which culture solution 1 or 2 was added, both Phospho-AKT (Ser473) and Phospho-AKT (Thr308) were activated.

Meanwhile, no reaction of Phospho-AKT (Ser473) was observed in WI-38 cells (fibroblasts) (i.e., normal cells) even when any of the aforementioned culture solutions was employed. That is, virtually no phosphorylation occurred at Ser473. Therefore, AKT was not activated.

### 10-4-2. ERK

FIG. 19 shows the degree of activation of ERK. The degree of activation of ERK was low only in the case of U251SP cells (glioma cells) to which culture solution 3 (i.e., solution irradiated with argon plasma) was added. In contrast, in U251SP cells (glioma cells) to which culture solution 1 or 2 was added, ERK was activated.

Meanwhile, slight Phospho-ERK reaction was observed in WI-38 cells (fibroblasts) (i.e., normal cells) even when any of the aforementioned culture solutions was employed. That is, virtually no phosphorylation of ERK occurred. Therefore, ERK was not activated.

### 10-5. Mechanism of Cancer Cell Killing in the Present Embodiment

This experiment indicated that activation of AKT or ERK was suppressed in U251SP cells (glioma cells). Thus, the plasma solution of the present embodiment suppresses activation of AKT or ERK in U251SP cells (glioma cells). Activation of AKT or ERK leads to inhibition of apoptosis of glioma cells. In this experiment, since activation of AKT or ERK was suppressed, apoptosis of glioma cells was promoted, leading to killing of U251SP cells (glioma cells). Therefore, conceivably, the plasma solution selectively kills only cancer cells while having virtually no effects on normal cells.

### 10-6. Effects of the Invention in Experiment E

The plasma solution of the present embodiment can suppress activation of both AKT and ERK. Thus, the plasma solution can suppress two signal transduction pathways of cancer cells, resulting in induction of apoptosis of the cancer cells.

Generally, many molecular target drugs among conventional anticancer agents act on specific factors; for example, such a molecular target drug acts only on AKT or ERK. However, actually, even when activation of only AKT is suppressed, cancer cells may be grown by using another signal transduction pathway (e.g., ERK). Therefore, the plasma solution of the present embodiment is envisaged to exhibit higher anticancer effect, as compared with conventional molecular target drugs. Also, the plasma solution is expected to exert its effect on a patient who has not been satisfactorily treated through administration of a conventional anticancer agent. In addition, the plasma solution of the present embodiment has virtually no effects on normal cells, and thus the plasma solution is considered to have few side effects. Furthermore, the plasma solution is expected to exert its effect on other types of cancer cells which are grown through activation of AKT or ERK.

### 10-7. Field of Application of the Evaluation Method of Experiment E

The method for evaluation of the plasma solution in this experiment may be applied to, for example, determination of the degree of AKT activity or ERK activity in cancer cells derived from a patient. On the basis of the difference between AKT activity and ERK activity in the cancer cells from the patient, an individual difference in the effects of the plasma solution can be evaluated. However, this application is only an example, and the present invention is not limited thereto.

### 11. EXPERIMENT F

### Argon-Hydrogen

### 11-1. Cells Employed

In this experiment, U251SP cells (glioma cells) shown in Table 6 were employed as cancer cells, and WI-38 cells (fibroblasts) shown in Table 6 were employed as normal cells.

### 11-2. Experimental Method

There were provided three types of culture media (i.e., inoculation of 1,000 cells, 5,000 cells, or 10,000 cells into a plate). Culturing was carried out for 24 hours. Components of the culture solution were the same as those employed in experiment A. Plasma irradiation was carried out according to the following three patterns.

| Type of plasma | | |
|---|---|---|
| (No plasma irradiation) | Irradiation time | Supplied gas |
| Argon plasma | 2 minutes | Ar |
| Argon-hydrogen plasma | 2 minutes | Ar + H₂ (H₂ gas: 1%) |

In this case, the amount of H₂ gas was 1% of the total amount of supplied gas. Thus, the experimental results correspond to a total of nine patterns. Cell number determination was carried out through MTS assay.

### 11-3. Experimental Results

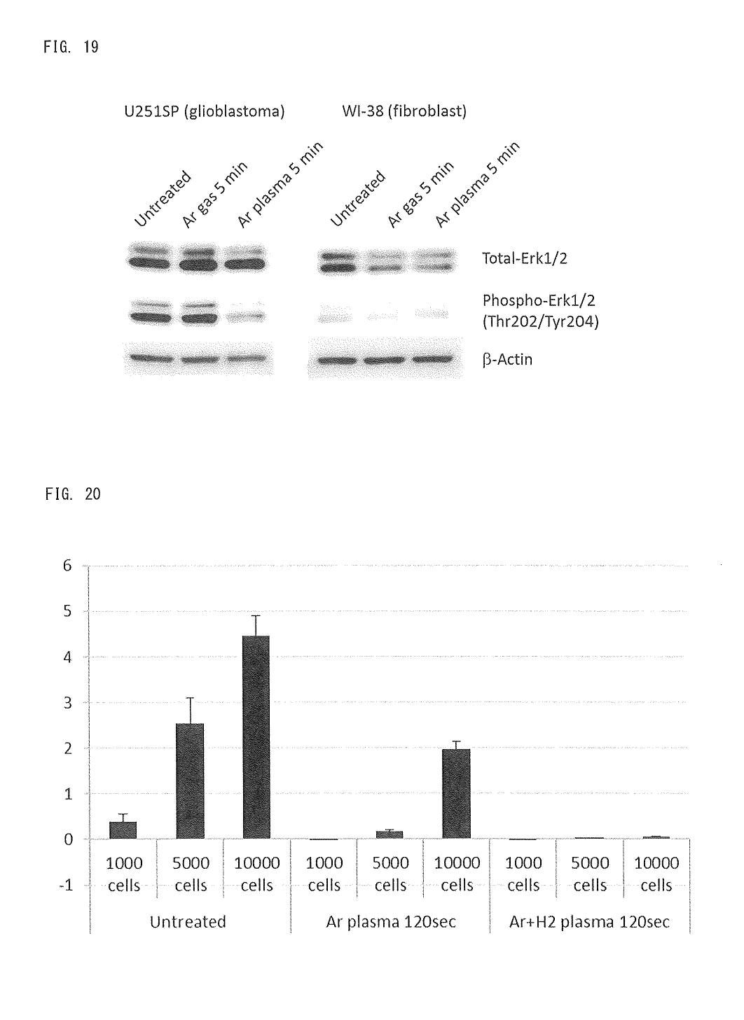

FIG. 20 is a graph showing the experimental results of the aforementioned nine patterns. As shown in FIG. 20, antitumor effect was observed in both cases of argon plasma and argon-hydrogen plasma. When 10,000 U251SP cells (glioma cells) were treated with a culture solution irradiated with argon plasma, about 40% of the U251SP cells survived. Meanwhile, when 10,000 U251SP cells (glioma cells) were treated with a culture solution irradiated with argon-hydrogen plasma, almost all the U251SP cells were killed.

FIG. 21 is a graph showing the results of a test for determining whether or not cancer cells can be selectively killed through argon-hydrogen plasma irradiation. WI-38 cells (fibroblasts) were also treated under the same conditions as those for U251SP cells (glioma cells), for comparison between the case of argon-hydrogen plasma irradiation and the case of no argon-hydrogen plasma irradiation. As shown in FIG. 21, when WI-38 cells (fibroblasts) (i.e., normal cells) were treated with a culture solution irradiated with argon-hydrogen plasma, virtually no cells were killed. The results of this experiment indicate that argon-hydrogen plasma irradiation achieves antitumor effect higher than that obtained through argon plasma irradiation. The selectivity of cancer cell killing in the case of argon-hydrogen plasma irradiation was almost equal to that in the case of argon plasma irradiation.

### 11-4. Effect of Argon-Hydrogen Plasma

Hydrogen radicals are generated by argon-hydrogen plasma. Conceivably, hydrogen radicals act in two different manners. In one conceivable manner, hydrogen radicals promote growth of cells. Conceivably, this cell growth occurs as a result of reduction of intracellular reactive oxygen species (ROS) with hydrogen radicals. In the other conceivable manner, hydrogen radicals provide cells with toxicity, since hydrogen radicals exhibit high reactivity. In this experiment, the effect of killing cancer cells was observed. However, cancer cells may fail to be killed under some experimental conditions.

### 12. EXPERIMENT G

### Culture Component and Antitumor Effect

In the aforementioned experiments, the plasma solution exhibits antitumor effect. The present inventors have first considered that radicals generated from atmospheric pressure plasma exhibit antitumor effect. However, the present inventors have had the idea that an antitumor substance exhibiting antitumor effect (i.e., selective killing of cancer cells) is produced through reaction between radicals generated from atmospheric pressure plasma and one or more components contained in a culture solution. Therefore, there was carried out an experiment for examining which component provides antitumor effect by irradiating any single-component aqueous solution with plasma.

### 12-1. Cells Employed

In this experiment, SKOV3 cells (ovarian cancer cells) shown in Table 8 were employed as cancer cells.

TABLE 8

| | |
|---|---|
| SKOV3 | Ovarian cancer cells |

### 12-2. Culture Component

RPMI 1640 was employed as a culture solution. Culture components thereof are shown in Table 9.

TABLE 9

Calcium nitrate•4H₂O
Magnesium sulfate (anhydrous)
Potassium chloride
Sodium hydrogen carbonate
Sodium chloride
Disodium phosphate (anhydrous)
L-Arginine
L-Asparagine (anhydrous)
L-Aspartic acid
L-Cystine•2HCl
L-Glutamic acid
L-Glutamine
Glycine
L-Histidine
Hydroxy-L-proline
L-Isoleucine
L-Leucine
L-Lysine•HCl
L-Methionine
L-Phenylalanine
L-Proline
L-Serine
L-Threonine
L-Tryptophan
L-Tyrosine•2Na•2H₂O
L-Valine
D-Biotin
Choline chloride
Folic acid
myo-Inositol
Niacinamide
p-Aminobenzoic acid
D-Pantothenic acid (hemicalcium)
Pyridoxine•HCl
Riboflavin
Thiamine•HCl
Vitamin B12
D-Glucose
Glutathione (reduced)
Phenol red•Na

In addition to the components shown above in Table 9, L-alanyl-L-glutamine, succinate.6H₂O.Na, succinic acid (free acid), choline bitartrate, or HEPES may be incorporated into the culture solution. However, such a component is not essential.

### 12-3. Preparation of Plasma Solution

The plasma solution employed in this experiment is prepared by irradiating a single-component aqueous solution with plasma, followed by addition of a culture solution to the aqueous solution, rather than by irradiating a culture solution with plasma. As used herein, the term “single-component aqueous solution” refers to an aqueous solution prepared by dissolving, in water, only one species of specific components shown in Table 9. The single-component aqueous solution may be, for example, an aqueous L-glutamine solution or an aqueous L-arginine solution.

Table 10 shows preparation steps of the plasma solution. Firstly, as shown in step 1 of Table 10, any one species of the components shown in Table 9 is dissolved in water, to thereby prepare a single-component aqueous solution. In this case, the single-component content of the aqueous solution is adjusted to become 10 times that of a common culture solution (RPMI 1640). In step 2, the single-component aqueous solution is allowed to stand for one hour. In step 3, the single-component aqueous solution is irradiated with plasma. Specifically, the single-component aqueous solution is irradiated with argon plasma employed in experiment A for five minutes. Other plasma irradiation conditions (e.g., irradiation distance) are the same as those employed in experiment A.

In step 4, a culture solution (RPMI 1640) is added to the single-component aqueous solution, to thereby prepare plasma solution 1. Thus, the single-component concentration of plasma solution 1 is 11 times that of the culture solution. In step 5, plasma solution 1 is subjected to filtration. In step 6, serum (FBS), sodium hydrogen carbonate, and D-glucose are added to plasma solution 1. In this experiment H, a plasma solution prepared through steps 1 to 6 was employed.

TABLE 10

| | |
|---|---|
| Step 1 | A single-component aqueous solution is prepared. |
| Step 2 | The single-component aqueous solution is allowed to stand for |
| | one hour. |
| Step 3 | The single-component aqueous solution is irradiated with |
| | plasma (Ar plasma for five minutes). |
| Step 4 | A culture solution (RPMI 1640) is added to the single-compo- |
| | nent aqueous solution, to thereby prepare plasma solution 1 |
| | (concentration: 11 times). |
| Step 5 | Plasma solution 1 is subjected to filtration. |
| Step 6 | FBS, sodium hydrogen carbonate, and D-glucose are added to |
| | plasma solution 1. |

### 12-4. Experimental Method

There were employed the aforementioned plasma solution 1 and plasma solution 2 (i.e., a solution prepared with water instead of a single-component aqueous solution). Plasma solution 2 was prepared by irradiating water with plasma, and adding a culture solution to the plasma-irradiated water. SKOV3 cells (ovarian cancer cells) were inoculated into a 96-well plate. Two types of samples were provided (number of cells contained in each sample: 5,000 or 10,000). Any one of plasma solution 1 and plasma solution 2 was added to SKOV3 cells (ovarian cancer cells). Cell viability for each sample was examined through MTS assay. The amount of a single-component aqueous solution prepared at one time in the aforementioned step 1 was 6 mL.

### 12-5. Experimental Results

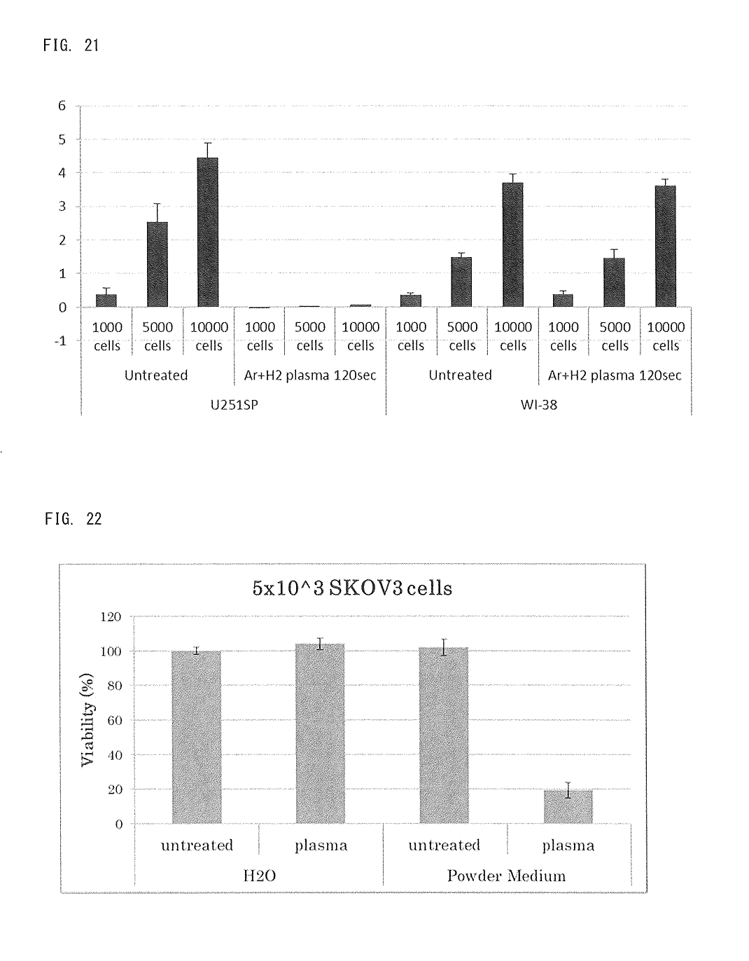

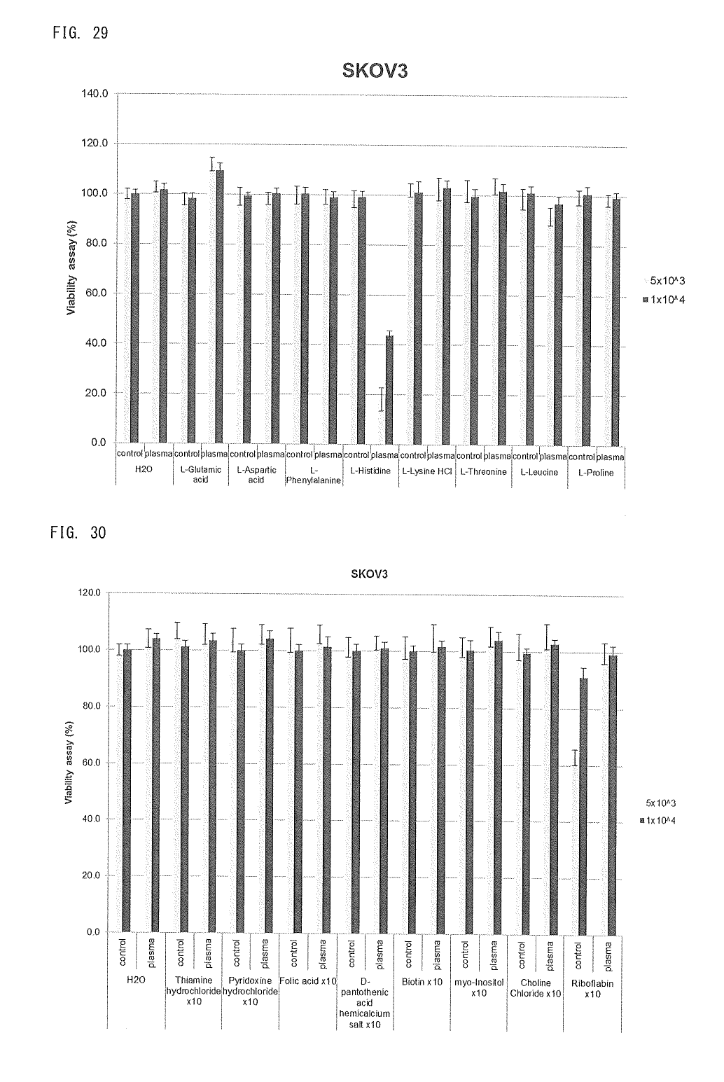

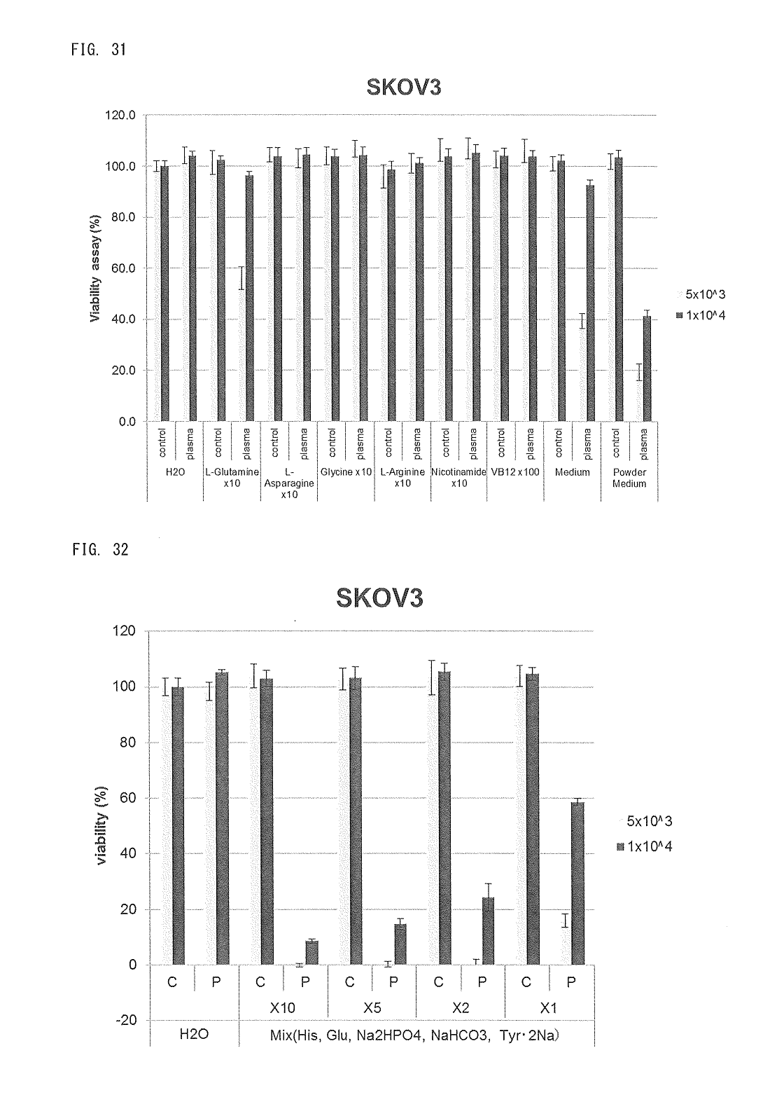

The experimental results are shown in FIGS. 22 to 36. The vertical axis of each graph corresponds to the viability of SKOV3 cells (ovarian cancer cells). In the case of a solution having no antitumor effect, the viability of SKOV3 cells (ovarian cancer cells) approximates 100%. Meanwhile, in the case of a solution having antitumor effect, the viability of SKOV3 cells (ovarian cancer cells) deviates from 100%. The lower the SKOV3 cell viability, the higher the antitumor effect.

The results of plasma solution 2 are shown on the left side of each of FIGS. 22 to 27. Plasma solution 2 does not have antitumor effect. Therefore, even when radicals or the like generated by atmospheric pressure plasma are supplied into water, a substance having antitumor effect is not generated in the water. Thus, conceivably, any substance having antitumor effect is generated through reaction between one or more culture components and radicals or the like.

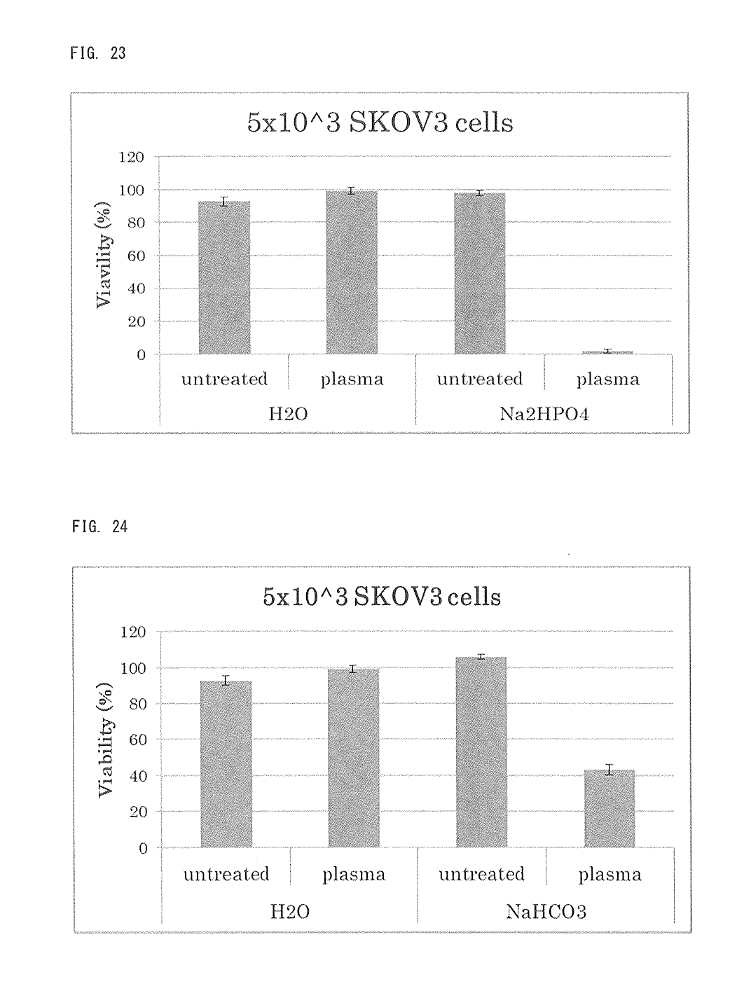

As shown in FIGS. 23 to 27, antitumor effect is exhibited by a plasma solution prepared by irradiating, with plasma, a single-component aqueous solution containing, as a solute, any of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O); and by adding a culture solution to the plasma-irradiated single-component aqueous solution.

As shown in FIG. 23, in the case of a plasma solution prepared by irradiating an aqueous disodium hydrogen phosphate (Na₂HPO₄) solution with plasma and adding a culture solution to the plasma-irradiated aqueous solution, the viability of 5,000 SKOV3 cells (ovarian cancer cells) was 5% or less.

As shown in FIG. 24, in the case of a plasma solution prepared by irradiating an aqueous sodium hydrogen carbonate (NaHCO₃) solution with plasma and adding a culture solution to the plasma-irradiated aqueous solution, the viability of 5,000 SKOV3 cells (ovarian cancer cells) was about 40%.

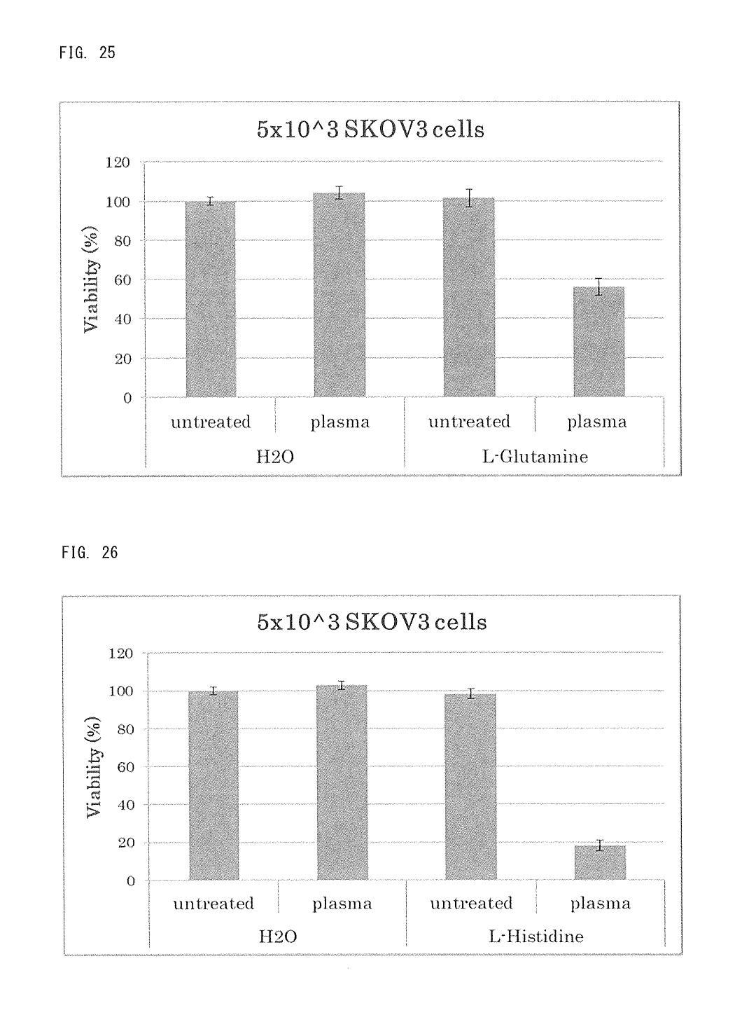

As shown in FIG. 25, in the case of a plasma solution prepared by irradiating an aqueous L-glutamine solution with plasma and adding a culture solution to the plasma-irradiated aqueous solution, the viability of 5,000 SKOV3 cells (ovarian cancer cells) was about 55%.

As shown in FIG. 26, in the case of a plasma solution prepared by irradiating an aqueous L-histidine solution with plasma and adding a culture solution to the plasma-irradiated aqueous solution, the viability of 5,000 SKOV3 cells (ovarian cancer cells) was about 20%.

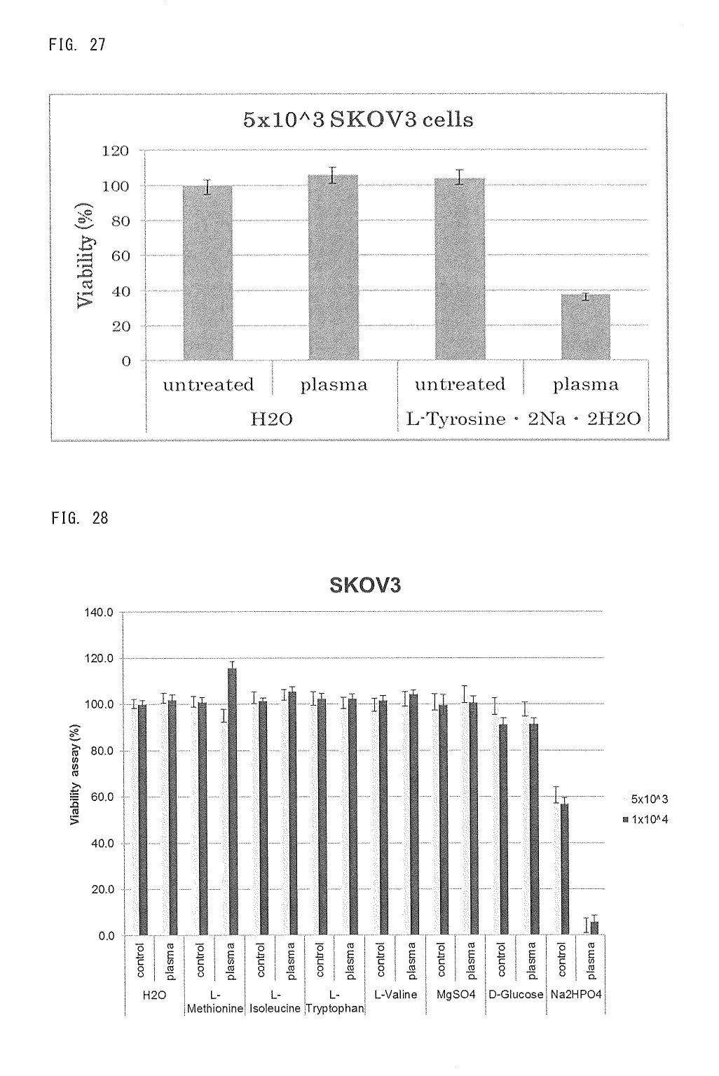

As shown in FIG. 27, in the case of a plasma solution prepared by irradiating an aqueous L-tyrosine disodium dihydrate (L-tyrosine.2Na.2H₂O) solution with plasma and adding a culture solution to the plasma-irradiated aqueous solution, the viability of 5,000 SKOV3 cells (ovarian cancer cells) was about 40%.

As shown in FIGS. 28 to 31, in the case of a plasma solution prepared by irradiating an aqueous solution containing a solute other than the aforementioned ones with plasma and adding a culture solution to the plasma-irradiated aqueous solution, the viability of 5,000 SKOV3 cells (ovarian cancer cells) was about 100%; i.e., no antitumor effect was observed.

FIG. 32 shows the results of an experiment for examining the antitumor effect of a plasma solution prepared by irradiating, with plasma, an aqueous solution containing, as solutes, the following five substances: disodium hydrogen phosphate (Na₂HPO₄), sodium hydrogen carbonate (NaHCO₃), L-glutamine, L-histidine, and L-tyrosine disodium dihydrate (L-tyrosine-2Na.2H₂O), each of which can serve as a raw material for a substance exhibiting antitumor effect; and by adding a culture solution to the plasma-irradiated aqueous solution.

As shown in FIG. 32, in the case of a plasma solution containing these five solutes (concentration: 11 times, which corresponds to “X10” in FIG. 32), the viability of 5,000 SKOV3 cells (ovarian cancer cells) was about 0%, and the viability of 10,000 SKOV3 cells (ovarian cancer cells) was about 10%. The lower the concentration of these solutes, the higher the viability of SKOV3 cells (ovarian cancer cells). These data suggest that the amount of these five solutes correlates to the amount of an antitumor substance generated through plasma irradiation.

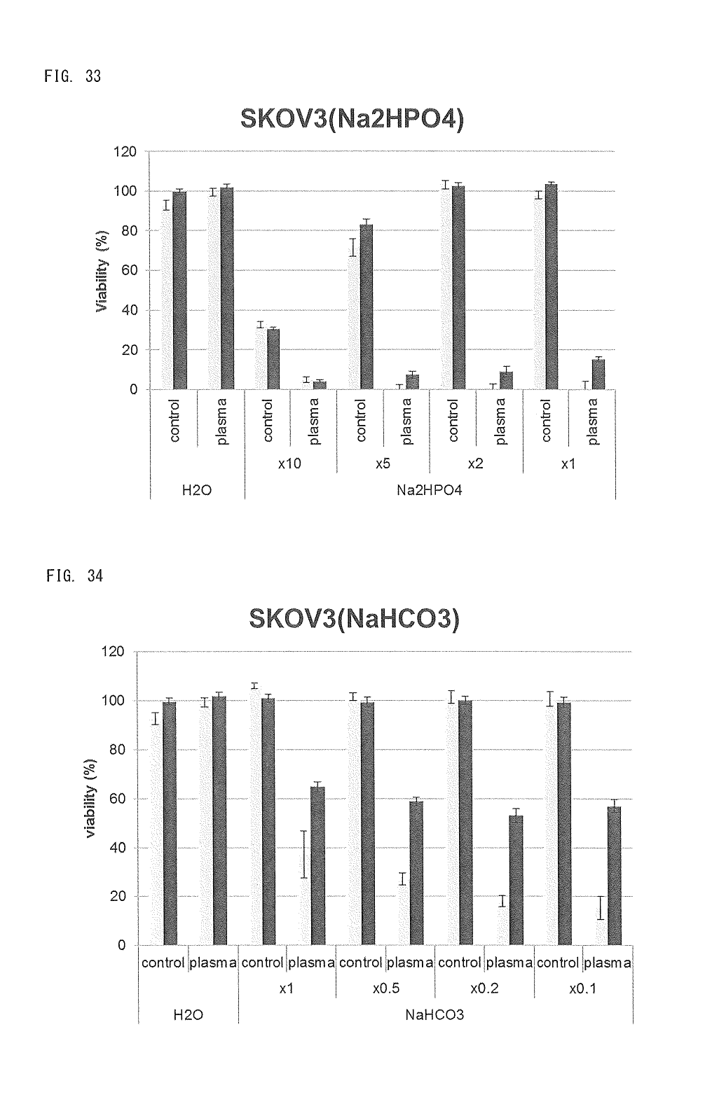

FIG. 33 is a graph showing the results of an experiment which was carried out by employing disodium hydrogen phosphate (Na₂HPO₄) in the same manner as shown in FIG. 32, and FIG. 34 is a graph showing the results of an experiment which was carried out by employing sodium hydrogen carbonate (NaHCO₃) in the same manner as shown in FIG. 32. Even when the concentration of any of these solutes was reduced, no great difference in viability of SKOV3 cells (ovarian cancer cells) was observed.

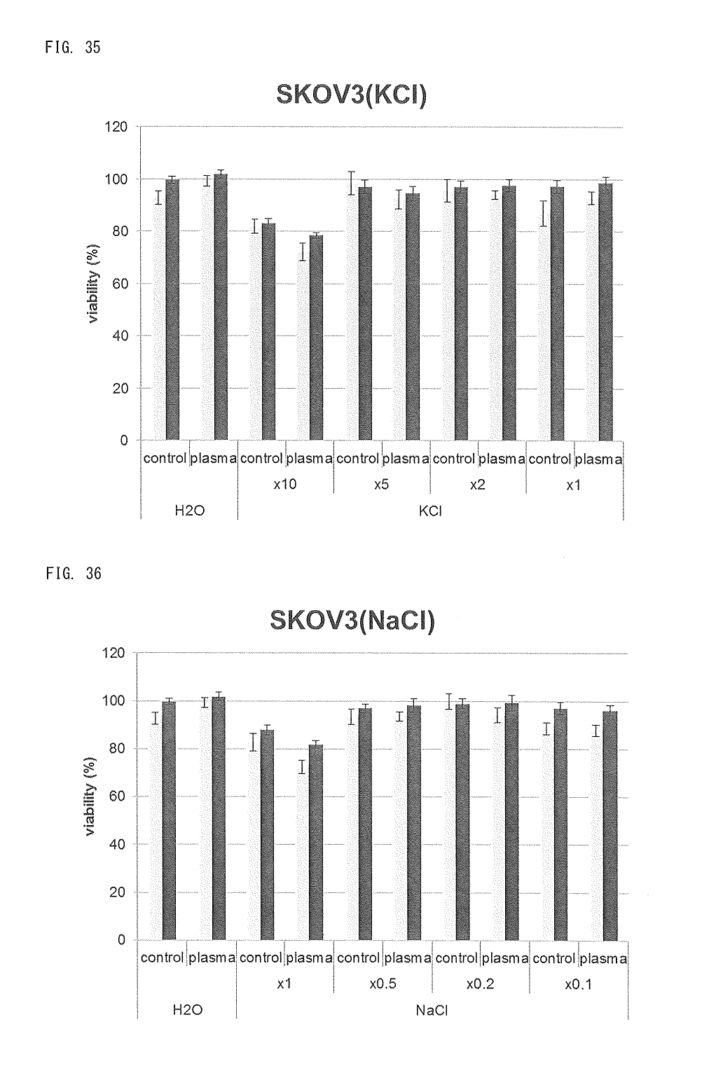

FIG. 35 is a graph showing the results of an experiment for examining the antitumor effect of a plasma solution prepared by irradiating, with plasma, an aqueous solution containing KCl (an inorganic salt) as a solute, and by adding a culture solution to the plasma-irradiated aqueous solution. FIG. 36 is a graph showing the results of an experiment for examining the antitumor effect of a plasma solution prepared in the same manner as described above (solute employed: NaCl (an inorganic salt)). As shown in FIGS. 35 and 36, these solutes (inorganic salts) cannot be employed as a raw material for an antitumor substance.

### 12-6. Experimental Discussion

As described above, antitumor effect is exhibited by a plasma solution prepared by irradiating any of the aforementioned five single-component aqueous solutions with plasma, and by adding a culture solution to the plasma-irradiated single-component aqueous solution. That is, a substance exhibiting antitumor effect is not necessarily generated from a single component. Each of the aforementioned amino acids and inorganic salts can serve as a raw material for a substance exhibiting antitumor effect. Thus, conceivably, any of these five substances reacts with certain radicals or the like supplied by plasma, to thereby generate a substance exhibiting antitumor effect through a multistage reaction.

### 13. EXPERIMENT H

### Anticancer-Agent-Resistant Cells

### 13-1. Cancer Cells Employed

In this experiment, there were employed, as shown in Table 11, common ovarian cancer cells, and ovarian cancer cells having resistance to an anticancer agent.

TABLE 11

| Name | Cell type | Presence or absence of resistance |
|---|---|---|
| NOS2 | Ovarian cancer cells | None |
| NOS2TR | Ovarian cancer cells | Paclitaxel resistance |
| NOS2CR | Ovarian cancer cells | Cisplatin resistance |
| NOS3 | Ovarian cancer cells | None |
| NOS3TR | Ovarian cancer cells | Paclitaxel resistance |
| NOS3CR | Ovarian cancer cells | Cisplatin resistance |

### 13-2. Experimental Method

Each type of ovarian cancer cells (10,000 cells) shown in Table 11 were inoculated into a 96-well plate and cultured in a common culture solution for 24 hours. Subsequently, the culture solution was exchanged with a plasma culture solution, and then culturing was carried out for 24 hours. Thereafter, ovarian cancer cell viability was evaluated through MTS assay. RPMI 1640 was employed as a culture solution. RPMI 1640 was irradiated with plasma. As in the case of experiment A, argon plasma irradiation was carried out according to the following three patterns: one-minute irradiation (60 seconds), two-minute irradiation (120 seconds), and three-minute irradiation (180 seconds).

### 13-3. Experimental Results

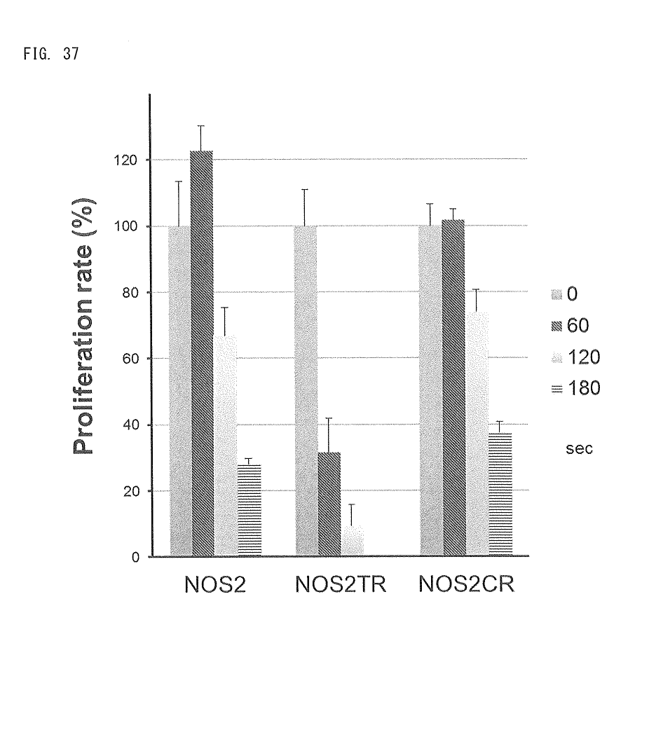

FIG. 37 shows the experimental results for NOS2 ovarian cancer cells. As shown in FIG. 37, antitumor effect was exhibited in the cases of NOS2 cells, NOS2TR cells, and NOS2CR cells. That is, the plasma solution of the present embodiment exhibits antitumor effect on cancer cells having resistance to an anticancer agent. Therefore, the plasma solution of the present embodiment exerts its effect on tumor having resistance to an anticancer agent. Particularly, the plasma solution of the present embodiment exhibited higher antitumor effect on NOS2TR cells than on NOS2 cells having no resistance to an anticancer agent.

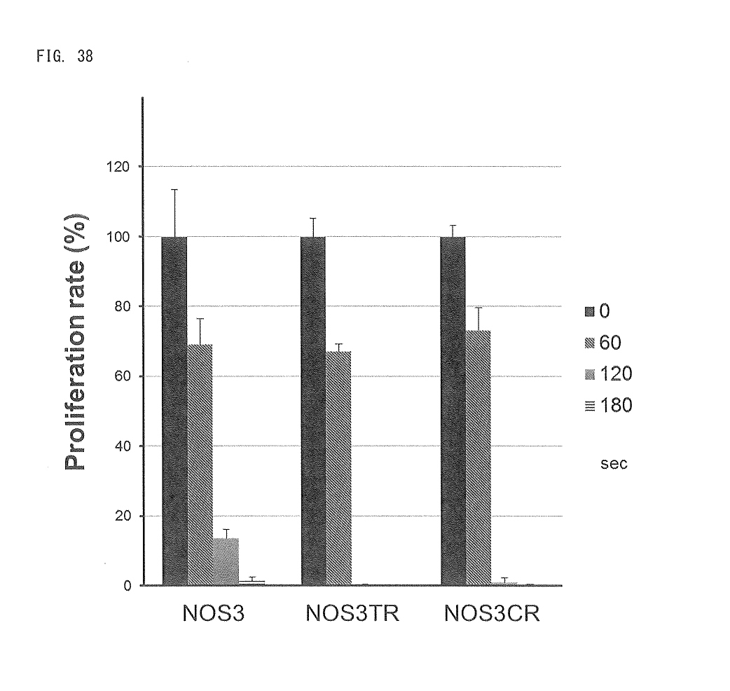

FIG. 38 shows the experimental results for NOS3 ovarian cancer cells. As shown in FIG. 38, antitumor effect was exhibited in the cases of NOS3 cells, NOS3TR cells, and NOS3CR cells. Specifically, the antitumor effect on NOS3 cells was comparable to that on NOS3TR cells or NOS3CR cells.

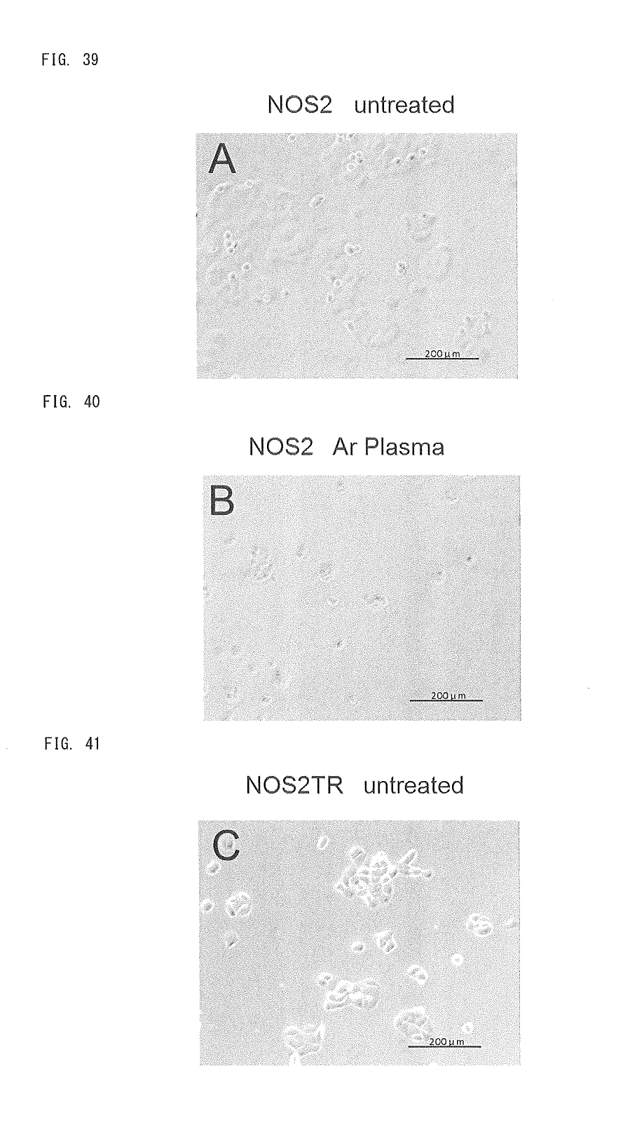

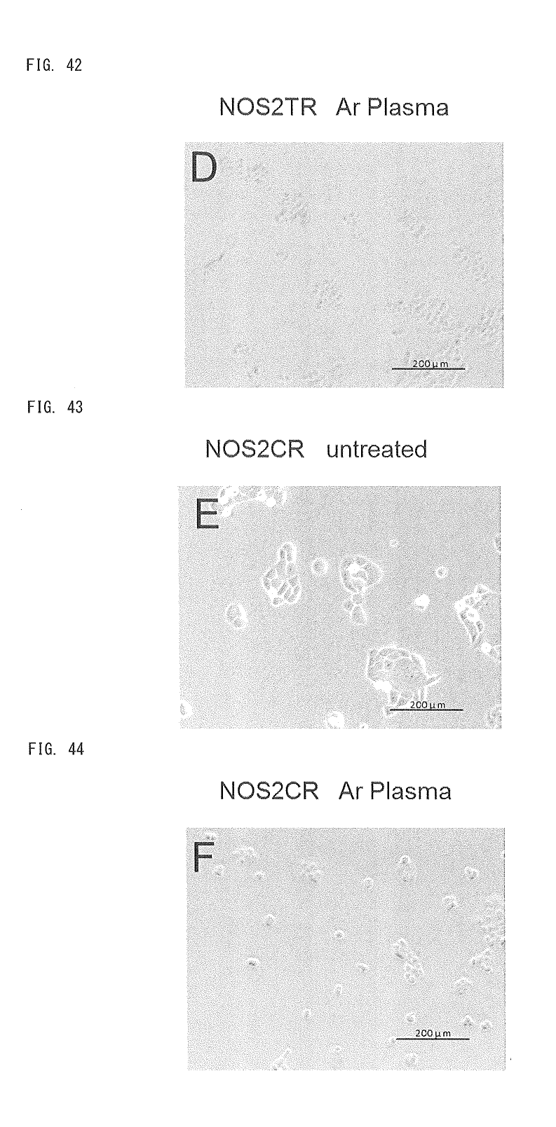

FIGS. 39 to 44 are micrographs of NOS2 ovarian cancer cells shown in Table 11. FIG. 39 is a micrograph showing NOS2 ovarian cancer cells cultured in a culture medium not irradiated with plasma. FIG. 40 is a micrograph showing NOS2 ovarian cancer cells cultured in a culture medium irradiated with plasma. FIG. 41 is a micrograph showing NOS2TR ovarian cancer cells cultured in a culture medium not irradiated with plasma. FIG. 42 is a micrograph showing NOS2TR ovarian cancer cells cultured in a culture medium irradiated with plasma. FIG. 43 is a micrograph showing NOS2CR ovarian cancer cells cultured in a culture medium not irradiated with plasma. FIG. 44 is a micrograph showing NOS2CR ovarian cancer cells cultured in a culture medium irradiated with plasma. As shown in these figures, ovarian cancer cells cultured in a plasma-irradiated culture medium (FIGS. 40, 42, and 44) are killed through apoptosis induction.

Thus, the plasma solution of the present embodiment can kill cancer cells having resistance to an anticancer agent. Conceivably, the reason for this is attributed to the fact that the plasma solution can block the signal transduction pathways of both AKT and ERK as described above.

### 14. EXPERIMENT I

### Animal Experiment: Anticancer Agent Resistance

### 14-1. Mice Employed

This experiment (animal experiment) was carried out by employing female nude mice. Any of two types of ovarian cancer cells (NOS2 cells or NOS2TR cells) were subcutaneously inoculated into both flank sites of each nude mouse. Specifically, 2,000 ovarian cancer cells were inoculated into each site, and the same amount of Matrigel was also administered thereto.

### 14-2. Experimental Method

From the next day following inoculation of ovarian cancer cells into the mice, a plasma culture solution was locally administered thrice a week. The plasma culture solution was prepared by irradiating SFM with argon plasma employed in experiment A. Specifically, SFM (3 mL) was irradiated with plasma for 10 minutes. The plasma culture solution (0.2 mL) was locally administered to each site inoculated with ovarian cancer cells. A culture solution not irradiated with plasma was injected into mice for comparison.

### 14-3. Experimental Results

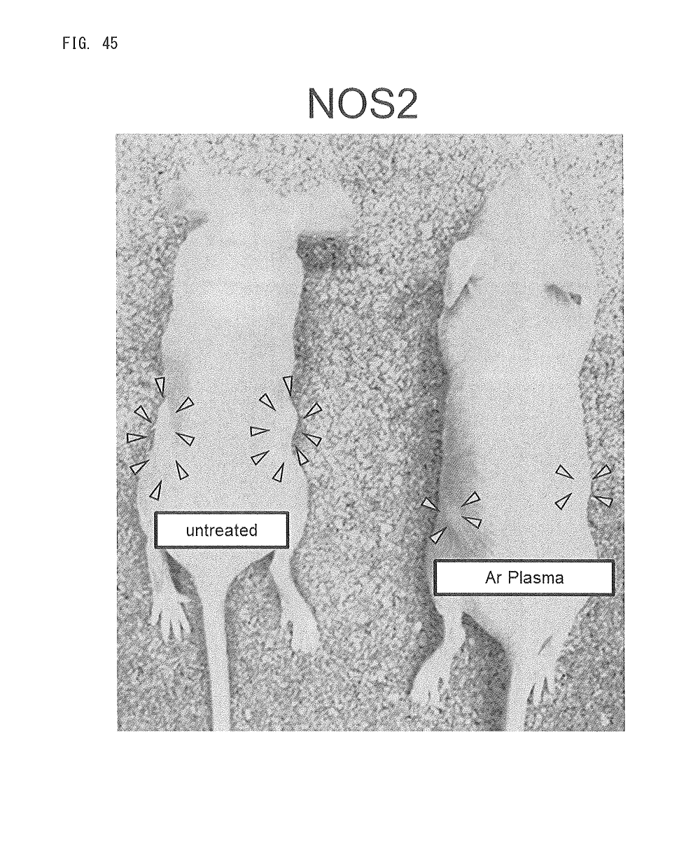

FIG. 45 is a photograph showing NOS2-inoculated mice (week 4). FIG. 45 (left side) shows a mouse to which a common culture solution was administered, and FIG. 45 (right side) shows a mouse to which the plasma culture solution was administered. In the mouse to which the common culture solution was administered, tumor-related swelling was observed, whereas in the mouse to which the plasma culture solution was administered, virtually no tumor-related swelling was observed.

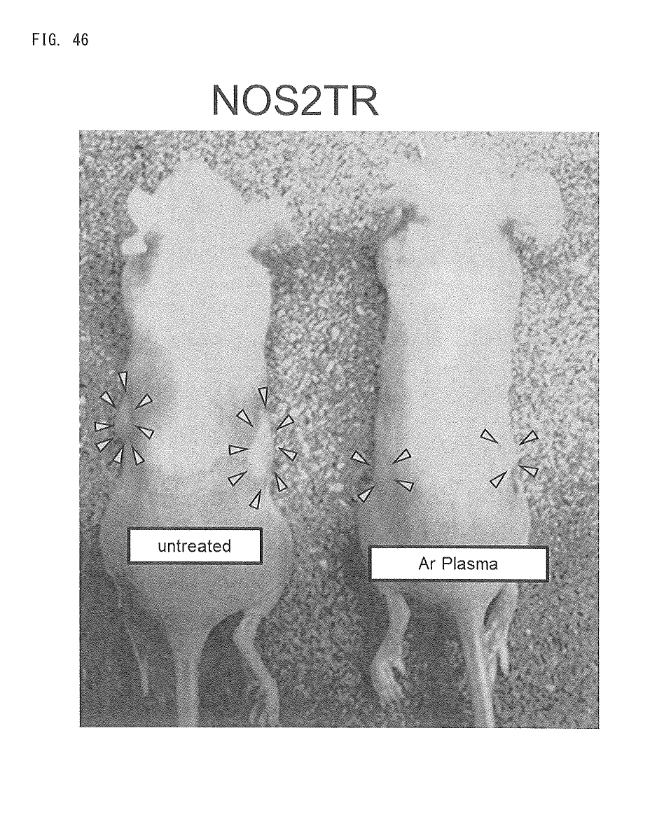

FIG. 46 is a photograph showing NOS2TR-inoculated mice (week 4). FIG. 46 (left side) shows a mouse to which a common culture solution was administered, and FIG. 46 (right side) shows a mouse to which the plasma culture solution was administered. Similar to the case of NOS2 inoculation shown in FIG. 45, in the mouse to which the common culture solution was administered, tumor-related swelling was observed, whereas in the mouse to which the plasma culture solution was administered, virtually no tumor-related swelling was observed.

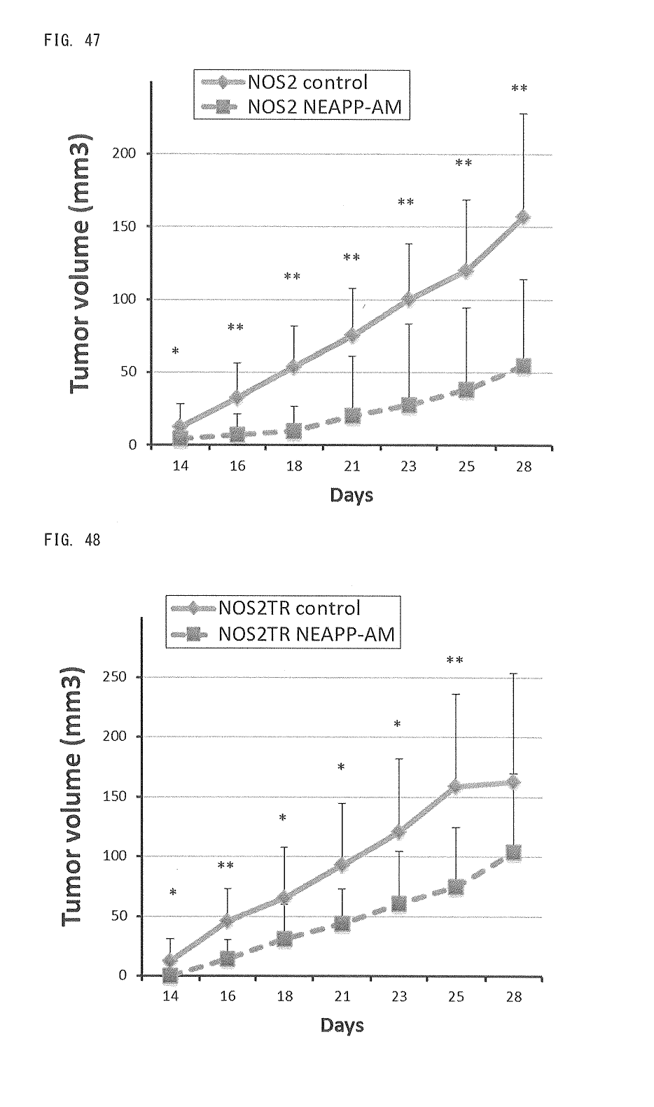

FIG. 47 is a graph showing a change in tumor volume in NOS2-inoculated mice. In FIG. 47, the horizontal axis corresponds to days after inoculation of ovarian cancer cells, and the vertical axis corresponds to the volume of ovarian cancer tumor. In FIG. 47, the solid line corresponds to data on the mice to which a common culture solution was administered, and the broken line corresponds to data on the mice to which the plasma culture solution was administered. As shown in FIG. 47, in the mice to which the plasma culture solution was administered, the volume of tumor was not so increased; i.e., tumor growth was suppressed, as compared with the mice to which the common culture solution was administered.

FIG. 48 is a graph showing a change in tumor volume in NOS2TR-inoculated mice (similar to FIG. 47). The data on NOS2TR-inoculated mice have a tendency similar to those on NOS2-inoculated mice.

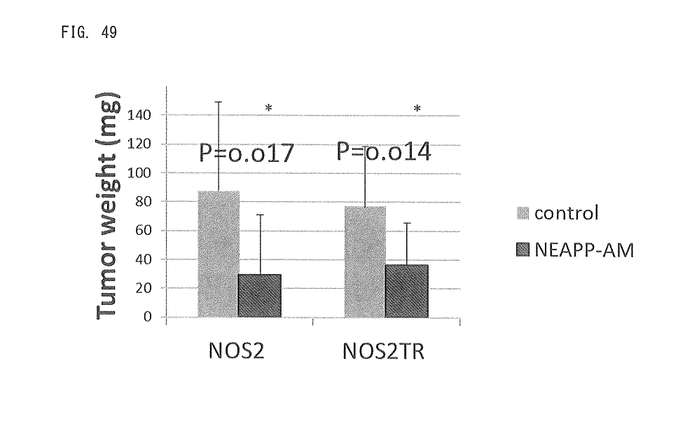

FIG. 49 is a graph showing the weight of tumor in mice 28 days after inoculation of ovarian cancer cells. In the NOS2-inoculated mice to which a common culture solution was administered, the weight of tumor was about 90 mg. In the NOS2-inoculated mice to which the plasma culture solution was administered, the weight of tumor was about 30 mg. In the NOS2TR-inoculated mice to which a common culture solution was administered, the weight of tumor was about 80 mg. In the NOS2TR-inoculated mice to which the plasma culture solution was administered, the weight of tumor was about 40 mg.

As described above, the antitumor effect of the plasma culture solution was also observed in the animal experiment employing nude mice.

### 15. SUMMARY OF THE PRESENT EMBODIMENT

As detailed above, the plasma solution of the present embodiment is prepared by irradiating a culture solution with plasma. Alternatively, the plasma solution is prepared by irradiating an aqueous solution containing a specific culture component (solute) with plasma, and then adding another culture component to the aqueous solution. The thus-prepared plasma solution exhibits antitumor effect. Also, the plasma solution exhibits the effect of killing cancer cells while killing virtually no normal cells. That is, the plasma solution can selectively kill cancer cells.

The plasma solution of the present embodiment is effective not only for cells, but also for living organisms. That is, the plasma solution serves as an anticancer agent which can induce apoptosis of only cancer cells for tumor reduction. Since the anticancer agent exhibits selectivity, it is expected to have virtually no side effects.

The present embodiment is only an example. Therefore, needless to say, various modifications and alterations may be made without departing from the scope of the present invention. Conceivably, the plasma solution of the present embodiment exerts its effect on, in addition to the cancer cells employed in the aforementioned experiments, a type of cancer which grows through activation of at least one signal transduction pathway of AKT and ERK. This is because, the plasma solution of the present embodiment induces apoptosis of only cancer cells by blocking the signal transduction pathways of both AKT and ERK.

Plasma conditions in the plasma irradiation device may be fed back through vacuum ultraviolet absorption spectroscopy. Thus, electron density, gas temperature, and oxygen radical density can be regulated.

### DESCRIPTION OF REFERENCE NUMERALS

100, 110: plasma irradiation device
10, 11: housing
10i, 11i: gas inlet port
10o, 11o: gas ejection port
2a, 2b: electrode
P: plasma region
H: hollow
P1: plasma irradiation device
M1: robot arm
PM: plasma solution production apparatus

1-17. (canceled)

18. A method for producing an antitumor aqueous solution for killing cancer cells, comprising:
an aqueous solution preparation step of preparing an aqueous solution through addition, to water, of a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O); and
a plasma irradiation step of irradiating the aqueous solution with atmospheric pressure plasma generated in a plasma generation region by means of a plasma generator.

19. An antitumor aqueous solution production method according to claim 18, wherein the plasma irradiation step employs a plasma density-time product of 1.2×10¹⁸ sec·cm⁻³ or more, the plasma density-time product being defined by the product of the plasma density in the plasma generation region and the time during which the aqueous solution is irradiated with the atmospheric pressure plasma.

20. An antitumor aqueous solution production method according to claim 18, which further comprises, after the plasma irradiation step, a culture component addition step of adding a culture component to the aqueous solution which has been irradiated with the atmospheric pressure plasma.

21. An antitumor aqueous solution production method according to claim 18, wherein, in the aqueous solution preparation step, a culture solution is prepared, as the aqueous solution, through addition of a culture component to water, and in the plasma irradiation step, the culture solution is irradiated with the atmospheric pressure plasma.

22. An antitumor aqueous solution production method according to claim 18, wherein, in the plasma irradiation step, the aqueous solution is irradiated with the atmospheric pressure plasma while the level of the aqueous solution is adjusted so that the aqueous solution is not exposed to the plasma generation region.

23. An antitumor aqueous solution production method according to claim 18, wherein the plasma generator includes a first electrode and a second electrode, the electrodes being located so as to face each other, and
in the plasma irradiation step, the aqueous solution is irradiated with the atmospheric pressure plasma while the first electrode and the second electrode are located outside the aqueous solution so that the aqueous solution is not provided between the electrodes.

24. An antitumor aqueous solution production method according to claim 18, wherein the antitumor aqueous solution selectively kills cancer cells.

25. An antitumor aqueous solution for killing cancer cells, produced by dissolving, in water, a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O), to thereby prepare an aqueous solution, and irradiating the aqueous solution with atmospheric pressure plasma.

26. An antitumor aqueous solution according to claim 25, wherein the atmospheric pressure plasma irradiation is carried out at a plasma density-time product of 1.2×10¹⁸ sec·cm⁻³ or more, the plasma density-time product being defined by the product of the plasma density in a plasma generation region of the atmospheric pressure plasma and the time during which the aqueous solution is irradiated with the atmospheric pressure plasma.

27. An antitumor aqueous solution according to claim 25, which is prepared by adding a culture component to the aqueous solution which has been irradiated with the atmospheric pressure plasma.

28. An antitumor aqueous solution according to claim 25, wherein the aqueous solution is a culture solution, and the culture solution is irradiated with the atmospheric pressure plasma.

29. An antitumor aqueous solution according to claim 25, which selectively kills cancer cells.

30. An antitumor aqueous solution according to claim 25, which induces apoptosis of cancer cells by blocking at least one signal transduction pathway of AKT and ERK of the cancer cells.

31. An antitumor aqueous solution according to claim 25, which kills cancer cells having resistance to an anticancer agent.

32. A method for producing an anticancer agent for killing cancer cells, the method comprising:
an aqueous solution preparation step of preparing an aqueous solution through addition, to water, of a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine.2Na.2H₂O); and
a plasma irradiation step of irradiating the aqueous solution with atmospheric pressure plasma generated in a plasma generation region by means of a plasma generator.

33. An anticancer agent for killing cancer cells, produced by dissolving, in water, a solute containing at least one of disodium hydrogen phosphate (Na₂HPO₄), sodium hydrogen carbonate (NaHCO₃), L-glutamine, L-histidine, and L-tyrosine disodium dihydrate (L-tyrosine 2Na.2H₂O), to thereby prepare an aqueous solution, and irradiating the aqueous solution with atmospheric pressure plasma, and the anticancer agent selectively kills cancer cells.
